(19) United States
(12) Patent Application Publication
(10) Pub. No.: US 2018/0228853 A1
(43) Pub. Date: Aug. 16, 2018

(54) COMPOSITION FOR TREATING AND PREVENTING VIRAL INFECTIONS

(71) Applicant: HSRX GROUP, LLC, Tucson, AZ (US)

(72) Inventors: Joshua M. COSTIN, Naples, FL (US); John M. Williams, Bonita Springs, FL (US); Dan LI, Fort Myers, FL (US)

(21) Appl. No.: 15/755,960

(22) PCT Filed: Aug. 31, 2016
(86) PCT No.: PCT/US16/49589

§ 371 Date: Feb. 27, 2018

Related U.S. Application Data
(60) Provisional application No. 62/212,339, filed on Aug. 31, 2015.

(51) Int. Cl. A61K 36/185; A61P 31/16; A61K 31/215; A61K 31/351; A61K 31/13; A61K 31/19; A61P 31/14

(52) U.S. Cl. CPC A61K 36/185; A61P 31/16; A61K 31/215; A61P 31/14; A61K 31/13; A61K 31/19; A61K 31/351

(57) ABSTRACT

The present invention relates generally to compositions and methods of use that include compounds that treat and prevent viral infections.

### CROSS-REFERENCE TO RELATED APPLICATIONS

This application claims the benefit of U.S. Provisional Application No. 62/212,339, filed on Aug. 31, 2015, the content of which is incorporated into the present application by reference.

### BACKGROUND OF THE INVENTION

### A. Field of the Invention

The present invention relates to formulations containing a mixture of compounds capable of preventing and treating viral infections.

### B. Description of Related Art

Viruses are relatively simple particles mainly made up of only a few proteins and nucleic acid that contains only a few genes; however, the proteins and nucleic acids can vary greatly between virus species. The variation in virus components leads to a great variation in diseases, complications, and symptoms of virus infections. Viral infections can cause conditions that vary from benign skin growths to failure of an infected person's immune system or bleeding, which when untreated can lead to death.

Development of anti-viral drugs is challenging. The great variation between viruses makes development of a general anti-viral treatment difficult. Further, viruses require a host cell for replication, hijacking the host cells own machinery to create viral particles; thus, treatments targeting the host dependent portion of a viral life cycle can often be harmful to the host organism. Moreover, many viral life cycles are very short in duration and many viruses have a high mutation rate, rendering treatments short lived in efficacy because of development of resistance. Currently, most anti-viral drugs target a specific subset of viruses, HIV, herpes virus, hepatitis B and C viruses, and influenza A and B viruses. There is also a great interest in developing drugs against Zika virus to battle the growing spread of Zika and the threat to unborn children. Unfortunately, these viruses only represent a very small fraction of life-threatening viruses and some of the treatments are already becoming obsolete as resistance develops. More anti-viral treatments and effective preventative measures are needed.

Many viruses that infect animals do have some things in common. Many of them, when budding from a host cell, envelope themselves in small portions of the host cell membrane. This envelope is typically made up of phospholipids and proteins from the host cell as well as some viral glycoproteins. The common components across all of the enveloped viruses, such as phospholipids or the high curvature of the virus envelope, may be a target that can be used to develop broad spectrum anti-viral drugs against all enveloped viruses. In addition, broad spectrum anti-viral drugs may target conserved amino acids, amino acid sequence motifs, and/or amino acid structural motifs of the viral glycoproteins. Further, a combination of active ingredients wherein the individual actives are effective against a few different viruses may in combination provide broad spectrum anti-viral protection.

Broad spectrum treatments against envelope viruses are of great interest, as such treatments may be effective against HIV, herpes simplex virus, Zika virus, dengue virus, and Influenza virus.

Interest in treating and preventing Zika virus, an enveloped virus of the flavivirus genus, has increased recently because of the rapid spread of the virus. Zika has been shown to cause Zika fever, which is rarely fatal to an adult, but when Zika infection is passed from a pregnant woman to her fetus, the fetus can develop birth defects that include microcephaly, defects of the eye, hearing deficits, and impaired growth. (Center for Disease Control and Prevention, Zika, 2016). Currently there is no vaccination for Zika and the best way to prevent Zika is to avoid mosquito bites. (Id.). Further, some antibodies against Zika may actually facilitate Zika virus infection of some cell types through antibody-dependent enhancement.

Infection by dengue virus, an enveloped virus of the flavivirus genus, is a leading cause of illness and death in the tropics and subtropics, where more than a third of the world's population resides. (Center for Disease Control and Prevention, Dengue, 2016). Dengue has been shown to cause dengue fever and dengue hemorrhagic fever (DHF). DHF may cause failure of the circulatory system and shock, and possibly death without prompt, appropriate treatment. (Id.). Currently there is no proven and effective treatment for dengue virus infection, but fluid replacement therapy may be useful in alleviating the symptoms of DHF. (Id.). Further, some antibodies against dengue virus actually facilitate dengue virus infection of some cell types through antibody-dependent enhancement.

Influenza is an acute respiratory illness caused by an influenza type A or B virus infection. (Nicholson et al. 1998). Influenza symptoms can include chills, cough, fatigue, fever, headache, muscle aches, and/or sore throat, with a severity ranging from mild symptoms that resemble a common cold, to typical “flu” like symptoms such as a combination of chills, cough, fatigue, fever, headache, muscle aches, and/or sore throat, to life-threatening symptoms including pneumonia and secondary bacterial infections. (U.S. Food and Drug Administration 2013; Kong 2009). Influenza morbidity rates in humans are high for all ages, but especially for children, the elderly, pregnant women, and patients with chronic illnesses. (Fields et al. 2001; Thompson et al. 2003).

Influenza pandemics are an unfortunately common occurrence, partly due to the continuous mutation of influenza viruses. In 2003, avian influenza (H5N1) infected over 400 humans and caused at least 258 deaths in 15 countries. (WHO, 2009). In 2009, a H1N1 swine flu outbreak in Mexico spread to more than 200 countries with over 18,000 deaths reported as of May 2010 and estimates of actual deaths worldwide of over 201,000. (WHO 2010; Dawood et al. 2012). Even in non-pandemic years, the death rate is high. In 2013, there were nearly 3,700 deaths associated with influenza in the United States alone. (Center for Disease Control and Prevention 2015).

Attempts to prevent influenza infections and pandemics include vaccination as well as antiviral drugs. (Kong 2009). Vaccinations are considered the most effective prevention tool; however, they are usually made to protect against only a few influenza viruses based on estimates of what viruses will be the most common during the upcoming season. (Subbarao et al. 2006; Center for Disease Control and Prevention 2014). Thus, though vaccination can be helpful, it may not be effective against viruses that were not foreseen to be the most common for the season. Currently, there are few anti-influenza drugs available for use. In the United States, only five anti-influenza drugs are approved: amantadine, rimantadine, oseltamivir, zanamivir, and peramivir. (Kong 2009; FDA 2014). However, pandemic influenza viruses are generally not sensitive to amantadine and rimantadine and viral resistance to the methods of actions for the approved drugs seems to be increasing. (Belshe et al. 1989; Hayden 1994; Le et al. 2005; Jefferson et al. 2006; Moscona 2005).

A folk medicine remedy for cold and flu, Sambucus nigra L. (elderberry), has been shown in clinical trials to be effective in treating influenzavirus A and B infections when taken as a syrup made from elderberry extract. (Roxas and Jurenka 2007; Zakay-Rones et al. 1995; 2004). These studies demonstrated that relatively large dosage amounts of the extract when compared with Tamiflu® (e.g., over 1000 times larger wt./vol.) could potentially reduce the duration of flu-like symptoms. The larger dosage amounts were achieved by increasing the frequency of treatments, which can lead to a decrease in patient compliance. Anti-influenza activities of some elderberry products are attributed to the presence of three flavonoids: averionol; tristenonol; and istrocyanidin. (Roschek and Alberte 2008). Extracts containing averionol, tristenonol, and istrocyanidin have been used to show that these three compounds may specifically bind some viruses, including some influenza strains, and may inhibit HIV. (US 2009/0149530). Further, US 2009/0149530 discloses that an unidentified active ingredient from an extract that may contain averionol, tristenonol, and istrocyanidin may inhibit infection of several viruses in vitro. (Id.).

### SUMMARY OF THE INVENTION

The present invention provides a solution to the current problems facing treatment and prevention of viral infections, including envelope virus infections, influenza, and influenza-like illness. The inventors have surprisingly found that a combination of several compounds found in elderberries can prevent and treat virus infection. The inventors have also found that specific relative concentrations of the compounds enhance the ability of the combined compounds to prevent and treat virus infection. In addition, the inventors have found that using compounds of the present invention with additional anti-viral drugs, such as anti-influenza compounds, enhance the ability of the combined compounds to prevent and treat viral infection.

In one aspect, disclosed is a composition of any one of, any combination of, or all of six biomarkers. In one instance the composition includes any one of, any combination of, or all of biomarker 1 having an accurate mass of 112.027 amu, biomarker 2 having an accurate mass of 126.032 amu, biomarker 3 having an accurate mass of 155.095 amu, biomarker 4 having an accurate mass of 160.087 amu, biomarker 5 having an accurate mass of 166.099 amu, and/or biomarker 8 having an accurate mass of 507.342 amu, wherein each biomarker is found in Sambucus nigra. The amounts of the ingredients within the composition can vary (e.g., amounts can be as low as 0.000001% to as high as 80% w/w or any range therein). In one instance biomarker 1 has a relative abundance of at least 2.36%, biomarker 2 has a relative abundance of at least 33.26%, biomarker 3 has a relative abundance of at least 1.86%, biomarker 4 has a relative abundance of at least 5.03%, biomarker 5 has a relative abundance of at least 9.26%, biomarker 8 has a relative abundance of at least 0.60%, wherein the relative abundance is relative abundance as compared to 0.01 mg/ml curcumin spiked in 1 mg/ml of the composition. In another instance, the composition includes at least 2, 3, 4, 5, or all of biomarkers 1 to 5 and 8.

In another aspect, disclosed is a composition that further includes any one of, or any combination of, or all of biomarker 6 having an accurate mass of 358.146 amu, biomarker 7 having an accurate mass of 478.295 amu, and biomarker 9 having an accurate mass of 606.436 amu, wherein each biomarker is found in Sambucus nigra. In one instance, biomarker 6 has a relative abundance of at least 11.37%, biomarker 7 has a relative abundance of at least 1.20%, and biomarker 9 has a relative abundance of at least 0.07%, wherein the relative abundance is relative abundance as compared to 0.01 mg/ml curcumin spiked in 1 mg/ml of the composition. In another instance, biomarker 1 has a relative abundance of between 2.36% and 6.94%, biomarker 2 has a relative abundance of between 33.26% and 85.75%, biomarker 3 has a relative abundance of between 1.86% and 4.69%, biomarker 4 has a relative abundance of between 5.03% and 12.89%, biomarker 5 has a relative abundance of between 9.26% and 24.11%, biomarker 8 has a relative abundance of between 0.60% and 1.75%, wherein the relative abundance is relative abundance as compared to 0.01 mg/ml curcumin spiked in 1 mg/ml of the composition. In another instance, biomarker 6 has a relative abundance of between 11.37% and 31.81%, biomarker 7 having a relative abundance of between 1.20% and 3.40%, and biomarker 9 having a relative abundance of between 0.07% and 1.38%, wherein the relative abundance is relative abundance as compared to 0.01 mg/ml curcumin spiked in 1 mg/ml of the composition. In yet another instance, the mass of each biomarker is the mass as determined by a Direct Analysis in Real Time-TOF (DART-TOF) mass spectrometer.

In another aspect, at least one of biomarkers 1 through 9 are synthetically obtained. In yet another aspect, at least one of biomarkers 1 through 9 are obtained from an organism. In one instance, at least one of biomarkers 1 through 9 are obtained from Sambucus nigra fruit. In another instance, the composition has at least 90%, preferably at least 95%, or at least 98% batch-to-batch chemical consistency of relative abundance for the biomarkers. In yet another instance, the composition further includes an anti-viral drug. In another instance, the composition includes an anti-influenza drug. In one instance, the anti-influenza drug is oseltamivir, zanamivir, rimantadine, amantadine, peramivir, or salts thereof, or any combination thereof.

In one aspect, the composition is formulated for oral administration. In one instance, the composition is one or more of a lozenge, a powder, a tablet, a gel-cap, a delayed release capsule, a quick release capsule, a gelatin, a liquid solution, and/or a dissolvable film. In another aspect, the composition is formulated for topical application, intravenous administration, and/or intranasal delivery. In one instance, the composition has an IC₅₀ lower than 500 μg/m1 against influenza virus. In another instance, at least one of biomarkers 1 to 5 and 8 is capable of binding to an influenza virus and blocking influenza viral entry into a cell. In yet another instance, at least one of biomarkers 1 to 5 and 8 is capable of binding hemagglutinin of the influenza virus.

In another aspect, the composition may further comprise one or more ingredients described herein. For example, the composition may comprise one or more additional ingredients selected from one or more pH adjusters, structuring agents, inorganic salts, and preservatives.

Also disclosed is a method of treating or preventing influenza and/or influenza-like illness in a subject, the method comprises administering any one of the compositions of the present invention to the subject. Further, there is disclosed a method of administering any one of the compositions of the present invention to a subject by administering any one of the compositions of the present invention to the subject. In a particular instance, the subject has been diagnosed with influenza and/or influenza-like illness.

In one aspect, disclosed is a method of treating a subject with influenza and/or influenza-like illness by administering any one of the compositions disclosed herein to the subject, wherein the subject is treated. In another instance, the subject has a fever, a headache, muscle aches, coughing, mucus discharge, or nasal congestion, or any combination thereof. In yet another instance, the influenza is caused by an Influenzavirus A and/or an Influenzavirus B virus. In one instance, the influenza virus is H1N1, H3N2, H3N5, H5N1, and/or Influenza B virus. In one instance, the influenza-like illness is caused by a rhinovirus. In another instance, the subject is administered a total sum of between 1 and 5,000 mg, preferably between 10 and 1,500 mg, between 50 and 1,000 mg, or between 100 and 500 mg of the biomarker(s) during a 24 hour period. In yet another instance, the composition is administered at least once a day for at least three days. In one instance, at least one of biomarkers 1 through 9 is synthetically obtained. In another instance, at least one of biomarkers 1 through 9 are obtained from an organism. In yet another instance, at least one of biomarkers 1 through 9 is obtained from Sambucus nigra fruit. In one instance, the composition has an at least 95% batch-to-batch chemical consistency of relative abundance for the biomarkers. In one instance, the composition further comprises an anti-viral drug. In another instance, the composition comprises an anti-influenza drug. In yet another instance, the anti-influenza drug is oseltamivir, zanamivir, rimantadine, amantadine, peramivir, or salts thereof, or any combination thereof. In one instance, the anti-influenza drug is oseltamivir, a salt thereof, or any combination thereof.

In another aspect, there is disclosed a method for treating a subject with influenza and/or influenza-like illness by administering any one of the compositions disclosed herein to the subject, wherein the composition is formulated for oral administration. In one instance, the composition is one or more of a lozenge, a powder, a tablet, a gel-cap, a delayed release capsule, a quick release capsule, a gelatin, a liquid solution, and/or a dissolvable film. In another instance, the composition is formulated for topical application, intravenous administration, and/or intranasal delivery. In yet another instance, the composition has an IC₅₀ lower than 500 μg/m1 against influenza virus. In one instance, at least one of biomarkers 1 to 5 and 8 is capable of binding to an influenza virus and blocking influenza viral entry into a cell. In another instance, at least one of biomarkers 1 to 5 and 8 is capable of binding hemagglutinin of the influenza virus.

In one aspect, there is disclosed a method for treating a subject infected with an envelope virus by administering any one of the compositions disclosed herein to the subject. In one instance, the subject is infected with a HIV, herpes complex virus, flavivirus virus, influenzavirus A virus, and/or influenzavirus B virus. In another instance, the subject is infected with a flavivirus virus and the flavivirus virus is Zika virus and/or dengue virus. In yet another instance, the subject is infected with Zika virus. In one instance, the subject is administered a total sum of between 1 and 5,000 mg, preferably between 10 and 1,500 mg, between 50 and 1,000 mg, or between 100 and 500 mg of the biomarker(s) during a 24 hour period. In another instance, the composition is administered at least once a day for at least three days.

In another aspect, there is disclosed a method for treating a subject infected with an envelope virus by administering any one of the compositions disclosed herein to the subject, wherein the composition is formulated for oral administration. In one instance, the composition is one or more of a lozenge, a powder, a tablet, a gel-cap, a delayed release capsule, a quick release capsule, a gelatin, a liquid solution, and/or a dissolvable film. In another instance, the composition is formulated for topical application, intravenous administration, and/or intranasal delivery. In yet another instance, the composition has an IC₅₀ lower than 500 μg/m1 against influenza virus. In one instance, at least one of biomarkers 1 to 5 and 8 is capable of binding to an influenza virus and blocking influenza viral entry into a cell. In another instance, at least one of biomarkers 1 to 5 and 8 is capable of binding hemagglutinin of the influenza virus.

In yet another aspect, there is disclosed a method of preventing influenza or influenza-like illness by administering any one of the compositions disclosed herein to the subject. In one instance, the influenza is caused by an Influenzavirus A and/or an Influenzavirus B virus. In another instance, the influenza virus is H1N1, H3N2, H3N5, H5N1, and/or Influenza B virus. In yet another instance, the influenza-like illness is caused by a rhinovirus. In yet another instance, the subject is administered a total sum of between 1 and 5,000 mg, preferably between 10 and 1,500 mg, between 50 and 1,000 mg, or between 100 and 500 mg of the biomarker(s) during a 24 hour period. In one instance, the composition is administered at least once a day for at least three days. In another instance, at least one of biomarkers 1 through 9 is synthetically obtained. In yet another instance, at least one of biomarkers 1 through 9 are obtained from an organism. In one instance, at least one of biomarkers 1 through 9 is obtained from Sambucus nigra fruit. In another instance, the composition has an at least 90%, preferably at least 95%, or at least 98% batch-to-batch chemical consistency of relative abundance for the biomarkers. In yet another instance, the composition further comprises an anti-influenza drug. In one instance, the anti-influenza drug is oseltamivir, zanamivir, rimantadine, amantadine, peramivir, or salts thereof, or any combination thereof. In another instance, the anti-influenza drug is oseltamivir, a salt thereof, or any combination thereof.

In one aspect, there is disclosed a method of preventing influenza and/or influenza-like illness by administering any one of the compositions disclosed herein to the subject, wherein the composition is formulated for oral administration. In one instance, the composition is one or more of a lozenge, a powder, a tablet, a gel-cap, a delayed release capsule, a quick release capsule, a gelatin, a liquid solution, and/or a dissolvable film. In another instance, the composition is formulated for topical application, intravenous administration, and/or intranasal delivery. In yet another instance, the composition has an IC₅₀ lower than 500 μg/m1 against influenza virus. In one instance, at least one of biomarkers 1 to 5 and 8 is capable of binding to an influenza virus and blocking influenza viral entry into a cell. In another instance, the at least one of biomarkers 1 to 5 and 8 is capable of binding hemagglutinin of the influenza virus.

In one aspect, there is disclosed a method for preventing infection of a subject by an envelope virus by administering any one of the compositions disclosed herein to the subject. In one instance, the envelope virus is a HIV, herpes complex virus, flavivirus virus, influenzavirus A virus, and/or influenzavirus B virus. In another instance, infection by a flavivirus virus infection is prevented, wherein the flavivirus virus is Zika virus and/or dengue virus. In yet another instance, the flavivirus is Zika virus. In yet another instance, the subject is administered a total sum of between 1 and 5,000 mg, preferably between 10 and 1,500 mg, between 50 and 1,000 mg, or between 100 and 500 mg of the biomarker(s) during a 24 hour period. In one instance, the composition is administered at least once a day for at least three days. In another instance, at least one of biomarkers 1 through 9 is synthetically obtained. In yet another instance, at least one of biomarkers 1 through 9 are obtained from an organism. In one instance, at least one of biomarkers 1 through 9 is obtained from Sambucus nigra fruit. In another instance, the composition has an at least 90%, preferably at least 95%, or at least 98% batch-to-batch chemical consistency of relative abundance for the biomarkers. In yet another instance, the composition further comprises an anti-influenza drug. In one instance, the anti-influenza drug is oseltamivir, zanamivir, rimantadine, amantadine, peramivir, or salts thereof, or any combination thereof. In another instance, the anti-influenza drug is oseltamivir, a salt thereof, or any combination thereof.

In one aspect, there is disclosed a method of preventing infection of a subject by an envelope virus by administering any one of the compositions disclosed herein to the subject, wherein the composition is formulated for oral administration. In one instance, the composition is one or more of a lozenge, a powder, a tablet, a gel-cap, a delayed release capsule, a quick release capsule, a gelatin, a liquid solution, and/or a dissolvable film. In another instance, the composition is formulated for topical application, intravenous administration, and/or intranasal delivery. In yet another instance, the composition has an IC₅₀ lower than 500 μg/m1 against influenza virus. In one instance, at least one of biomarkers 1 to 5 and 8 is capable of binding to an influenza virus and blocking influenza viral entry into a cell. In another instance, the at least one of biomarkers 1 to 5 and 8 is capable of binding hemagglutinin of the influenza virus.

In another aspect, there is disclosed a method of producing any one of the compositions disclosed herein by producing a composition having an at least 90%, preferably at least 95% or at least 98% batch-to-batch chemical consistency of relative abundance for the biomarkers.

In some aspects of the invention, the composition may further comprise one or more nutraceutical and/or pharmaceutically acceptable carriers or diluents. These carriers/diluents can be adjuvants, excipients, or vehicles such as preserving agents, fillers, disintegrating agents, wetting agents, emulsifiers, suspending agents, sweeteners, flavorings, fragrance, antibacterial agents, antifungal agents, lubricating agents, vitamins, polymers, siloxane containing compounds, essential oils, structuring agents, and dispensing agents. Each carrier is acceptable in the sense of being compatible with the other ingredients of the formulation and not injurious to the subject. In some aspects of the invention, the carrier can include at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Non-limiting examples of diluents/carriers are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

The composition can be stored for one month, 6 months, 12 months, 18 months, or 24 months at room temperature. In some aspects of the invention, the composition is formulated as a powder, a tablet, a gel-cap, a bead, an edible tablet, a dissolvable film, a liquid capable of being dispersed through the air, a gelatin, a lotion, a transdermal patch, or a liquid solution for oral administration. In some aspects of the invention, the formulated composition can be comprised in a solid nanoparticle, a lipid-containing nanoparticle, a lipid-based carrier, a sealed conduit, a straw, sealed bag, or any combination thereof. In other aspects of the invention, the composition can be formulated for administration by injection.

Kits that include the compositions of the present invention are also contemplated. In certain embodiments, the composition is comprised in a container. The container can be a bottle, dispenser, package, or a straw. The container can dispense a predetermined amount of the composition. In certain aspects, the compositions are dispensed as a pill, a tablet, a capsule, a transdermal patch, an edible chew, a cream, a lotion, a gel, spray, mist, dollop, a powder, or a liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

Also contemplated is a product that includes the composition of the present invention. In non-limiting aspects, the product can be a nutraceutical product. The nutraceutical product can be those described in other sections of this specification or those known to a person of skill in the art. In other non-limiting aspects, the product can be a pharmaceutical product. The pharmaceutical and/or nutraceutical product can be those described in other sections of this specification or those known to a person of skill in the art. Non-limiting examples of products include a pill, a tablet, an edible chew, a capsule, a cream, a lotion, a gel, a spray, a mist, a dissolving film, a transdermal patch, or a liquid, etc.

Also disclosed are the following Embodiments 1 to 47 of the present invention. Embodiment 1 is a composition comprising any one of, or any combination of, the following biomarkers: (a) biomarker 1 having an accurate mass of 112.027 amu and having a relative abundance of at least 2.36%; (b) biomarker 2 having an accurate mass of 126.032 amu and having a relative abundance of at least 33.26%; (c) biomarker 3 having an accurate mass of 155.095 amu and having a relative abundance of at least 1.86%; (d) biomarker 4 having an accurate mass of 160.087 amu and having a relative abundance of at least 5.03%; (e) biomarker 5 having an accurate mass of 166.099 amu and having a relative abundance of at least 9.26%; or (f) biomarker 8 having an accurate mass of 507.342 amu and having a relative abundance of at least 0.60%, wherein each biomarker is found in Sambucus nigra, and wherein the relative abundance is relative abundance as compared to 0.01 mg/ml curcumin spiked in 1 mg/ml of the composition. Embodiment 2 is the composition of Embodiment 1, having at least 2, 3, 4, 5, or all of biomarkers 1 to 5 and 8. Embodiment 3 is the composition of any one of Embodiments 1 to 2, wherein the composition further comprises any one of, or any combination of, or all of the following additional biomarkers: (g) biomarker 6 having an accurate mass of 358.146 amu; (h) biomarker 7 having an accurate mass of 478.295 amu; (i) biomarker 9 having an accurate mass of 606.436 amu, wherein each biomarker is found in Sambucus nigra. Embodiment 4 is the composition of Embodiment 3, further comprising: (j) biomarker 6 having a relative abundance of at least 11.37%; (k) biomarker 7 having a relative abundance of at least 1.20%; and (1) biomarker 9 having a relative abundance of at least 0.07%, wherein the relative abundance is relative abundance as compared to 0.01 mg/ml curcumin spiked in 1 mg/ml of the composition. Embodiment 5 is the composition of Embodiment 1, further comprising: (a) biomarker 1 having a relative abundance of between 2.36% and 6.94%; (b) biomarker 2 having a relative abundance of between 33.26% and 85.75%; (c) biomarker 3 having a relative abundance of between 1.86% and 4.69%; (d) biomarker 4 having a relative abundance of between 5.03% and 12.89%; (e) biomarker 5 having a relative abundance of between 9.26% and 24.11%; (f) biomarker 8 having a relative abundance of between 0.60% and 1.75%, wherein the relative abundance is relative abundance as compared to 0.01 mg/ml curcumin spiked in 1 mg/ml of the composition. Embodiment 6 is the composition of Embodiment 4, further comprising: (j) biomarker 6 having a relative abundance of between 11.37% and 31.81%; (k) biomarker 7 having a relative abundance of between 1.20% and 3.40%; and (1) biomarker 9 having a relative abundance of between 0.07% and 1.38%, wherein the relative abundance is relative abundance as compared to 0.01 mg/ml curcumin spiked in 1 mg/ml of the composition. Embodiment 7 is the composition of any of Embodiments 1 to 6 wherein the mass of each biomarker is the mass as determined by a Direct Analysis in Real Time-TOF (DART-TOF) mass spectrometer. Embodiment 8 is the composition of any one of Embodiments 1 to 7, wherein at least one of biomarkers 1 through 9 are synthetically obtained. Embodiment 9 is the composition of any one of Embodiments 1 to 7, wherein at least one of biomarkers 1 through 9 are obtained from an organism. Embodiment 10 is the composition of Embodiment 9, wherein at least one of biomarkers 1 through 9 are obtained from Sambucus nigra fruit. Embodiment 11 is the composition of any one of Embodiments 1 to 10, wherein the composition has an at least 90%, preferably at least 95%, or at least 98% batch-to-batch chemical consistency of relative abundance for the biomarkers. Embodiment 12 is the composition of any one of Embodiments 1 to 11, wherein the composition further comprises an anti-viral drug. Embodiment 13 is the composition of Embodiment 12, wherein the composition further comprises an anti-influenza drug. Embodiment 14 is the composition of Embodiment 13, wherein the anti-influenza drug is oseltamivir, zanamivir, rimantadine, amantadine, peramivir, or salts thereof, or any combination thereof. Embodiment 15 is the composition of Embodiment 14, wherein the anti-influenza drug is oseltamivir, a salt thereof, or any combination thereof. Embodiment 16 is the composition of any one of Embodiments 1 to 15, wherein the composition is formulated for oral administration. Embodiment 17 is the composition of Embodiment 16, wherein the composition is one or more of a lozenge, a powder, a tablet, a gel-cap, a delayed release capsule, a quick release capsule, a gelatin, a liquid solution, and/or a dissolvable film. Embodiment 18 is the composition of any one of Embodiments 1 to 15, wherein the composition is formulated for topical application, intravenous administration, and/or intranasal delivery. Embodiment 19 is the composition of any one of Embodiments 1 to 18, wherein the composition has an IC50 lower than 500 μg/m1 against influenza virus. Embodiment 20 is the composition of any of Embodiments 1 to 19, wherein at least one of biomarkers 1 to 5 and 8 is capable of binding to an influenza virus and blocking influenza viral entry into a cell. Embodiment 21 is the composition of Embodiment 20, wherein the at least one of biomarkers 1 to 5 and 8 is capable of binding hemagglutinin of the influenza virus. Embodiment 22 is a method of treating a subject having influenza and/or an influenza-like illness, the method comprising administering any one of the compositions of Embodiments 1 to 21 to the subject, wherein the subject is treated. Embodiment 23 is the method of Embodiment 22, wherein the subject has a fever, a headache, muscle aches, coughing, mucus discharge, or nasal congestion, or any combination thereof. Embodiment 24 is the method of any one of Embodiments 22 to 23, wherein the subject has influenza and is infected with an Influenzavirus A and/or an Influenzavirus B virus. Embodiment 25 is the method of Embodiment 24, wherein the influenza virus is H1N1, H3N2, H3N5, H5N1, and/or Influenza B virus. Embodiment 26 is the method of any one of Embodiments 22 to 23, wherein the subject has an influenza-like illness and is infected with Rhinovirus. Embodiment 27 is the method of any one of Embodiments 22 to 26, wherein the subject is administered a total sum of between 1 and 5,000 mg, preferably between 10 and 1,500 mg, between 50 and 1,000 mg, or between 100 and 500 mg of the biomarker(s) during a 24 hour period. Embodiment 28 is the method of any one of Embodiments 22 to 27, wherein the composition is administered at least once a day for at least three days. Embodiment 29 is a method of treating a subject infected with an envelope virus, the method comprising administering any one of the compositions of Embodiments 1 to 21 to the subject, wherein the subject is treated. Embodiment 30 is the method of Embodiment 29, wherein the subject is infected with a HIV, herpes complex virus, flavivirus virus, influenzavirus A virus, and/or influenzavirus B virus. Embodiment 31 is the method of any one of Embodiments 29 to 30, wherein the subject is infected with a flavivirus virus and the flavivirus virus is Zika virus and/or dengue virus. Embodiment 32 is the method of Embodiment 31, wherein the subject is infected with Zika virus. Embodiment 33 is the method of any one of Embodiments 29 to 32, wherein the subject is administered a total sum of between 1 and 5,000 mg, preferably between 10 and 1,500 mg, between 50 and 1,000 mg, or between 100 and 500 mg of the biomarker(s) during a 24 hour period. Embodiment 34 is the method of any one of Embodiments 29 to 33, wherein the composition is administered at least once a day for at least three days. Embodiment 35 is a method of preventing influenza and/or an influenza-like illness in a subject, the method comprising administering any one of the compositions of Embodiments 1 to 21 to the subject, wherein influenza and/or an influenza-like illness is prevented. Embodiment 36 is the method of Embodiment 35, wherein influenza caused by an Influenzavirus A and/or an Influenzavirus B virus is prevented. Embodiment 37 is the method of Embodiment 36, wherein the influenza virus is H1N1, H3N2, H3N5, H5N1, and/or Influenza B virus. Embodiment 38 is the method of Embodiment 35, wherein influenza-like illness caused by a Rhinovirus is prevented. Embodiment 39 is the method of any one of Embodiments 35 to 38, wherein the subject is administered a total sum of between 1 and 5,000 mg, preferably between 10 and 1,500 mg, between 50 and 1,000 mg, or between 100 and 500 mg of the biomarker(s) during a 24 hour period. Embodiment 40 is the method of any one of Embodiments 35 to 39, wherein the composition is administered at least once a day for at least three days. Embodiment 41 is a method of preventing an envelope virus infection in a subject, the method comprising administering any one of the compositions of Embodiments 1 to 21 to the subject, wherein an envelope virus infection is prevented. Embodiment 42 is the method of Embodiment 41, wherein the envelope virus infection that is prevented is an infection by a HIV, herpes complex virus, flavivirus virus, influenzavirus A virus, and/or influenzavirus B virus. Embodiment 43 is the method of any one of Embodiments 41 to 42, wherein a flavivirus virus infection is prevented and the flavivirus virus is Zika virus and/or dengue virus. Embodiment 44 is the method of Embodiment 43, wherein a Zika virus infection is prevented. Embodiment 45 is the method of any one of Embodiments 41 to 44, wherein the subject is administered a total sum of between 1 and 5,000 mg, preferably between 10 and 1,500 mg, between 50 and 1,000 mg, or between 100 and 500 mg of the biomarker(s) during a 24 hour period. Embodiment 46 is the method of any one of Embodiments 41 to 45, wherein the composition is administered at least once a day for at least three days. Embodiment 47 is a method of producing a composition of any of Embodiments 1 through 21, wherein the method of producing produces a composition having an at least 90%, preferably at least 95% or at least 98% batch-to-batch chemical consistency of relative abundance for the biomarkers.

“Therapeutic agent” encompasses the compounds specifically claimed herein. It also encompasses such compounds together with nutraceutical and/or pharmaceutically acceptable salts thereof. Useful salts are known to those skilled in the art and include salts with inorganic acids, organic acids, inorganic bases, or organic bases. Therapeutic agents useful in the present invention are those compounds that affect a desired, beneficial, and often pharmacological, effect upon administration to a human or an animal, whether alone or in combination with other nutraceutical and/or pharmaceutical excipients or inert ingredients.

The term “biomarker” refers to the compound defined as the biomarker, analogues thereof, derivatives thereof, or salt forms of any analogue or derivative thereof.

The term “accurate mass” refers to a measured mass of a molecule experimentally determined for an ion of known charge. The units for accurate mass include atomic mass units (amu) and milli unified atomic mass units (mmu). The term “molecular weight” refers to the average weight of the molecule with all of the different isotopic compositions present in a compound but weighted for their natural abundance.

The term “relative abundance” refers to the abundance of a compound of interest relative to the abundance of a reference compound. In particular aspects, relative abundance is the raw intensity of a mass spectrometry peak for the compound of interest over the raw intensity of a mass spectrometry peak for a reference compound. In one non-limiting instance, the mass spectrometry peaks can be obtained by the use of DART-TOF mass spectrometry. In another particular aspect, the reference compound is a compound that is spiked, or doped, into a sample containing the compound of interest. In yet another particular aspect, the reference compound is a compound that does not exist in the sample previous to its addition to the sample for determining relative abundance. In another particular aspect, the reference compound can be curcumin.

Accurate mass and relative abundances described herein are based on experiments using particular instruments and particular settings and can change from instrument to instrument. There is variability in each measurement. Thus, the accurate mass and relative abundances are defined as being close to as understood by one of ordinary skill in the art. In one non-limiting embodiment the terms are defined to be within 20%, preferably 10%, preferably within 5%, more preferably within 1%, and most preferably within 0.5%. In one non-limiting embodiment, the accurate mass has an error of within +/−20 mmu, preferably 10 mmu, more preferably within 5 mmu, and most preferably within 1 mmu. In one non-limiting embodiment, the relative abundance has an error of +/−20%, preferably 10%, preferably within 5%, and more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

“Patient,” “subject,” or “individual” refers to a mammal (e.g., human, primate, dog, cat, bovine, ovine, porcine, equine, mouse, rat, hamster, rabbit, or guinea pig). In particular aspects, the patient, subject, or individual is a human.

“Inhibiting” or “reducing” or any variation of these terms includes any measurable decrease or complete inhibition to achieve a desired result.

“Effective” or “treating” or “preventing” or any variation of these terms means adequate to accomplish a desired, expected, or intended result.

“Analogue” and “analog,” when referring to a compound, refers to a modified compound wherein one or more atoms have been substituted by other atoms, or wherein one or more atoms have been deleted from the compound, or wherein one or more atoms have been added to the compound, or any combination of such modifications. Such addition, deletion or substitution of atoms can take place at any point, or multiple points, along the primary structure comprising the compound.

“Derivative,” in relation to a parent compound, refers to a chemically modified parent compound or an analogue thereof, wherein at least one substituent is not present in the parent compound or an analogue thereof. One such non-limiting example is a parent compound which has been covalently modified. Typical modifications are amides, carbohydrates, alkyl groups, acyl groups, esters, pegylations and the like.

A “therapeutically equivalent” compound is one that has essentially the same effect in the treatment of a disease or condition as one or more other compounds. A compound that is therapeutically equivalent may or may not be chemically equivalent, bioequivalent, or generically equivalent.

“Parenteral injection” refers to the administration of small molecule drugs via injection under or through one or more layers of skin or mucus membranes of an animal, such as a human.

“Bioavailability” refers to the extent to which the therapeutic agent absorbed from the formulation.

“Systemic,” with respect to delivery or administration of a therapeutic agent to a subject, indicates that the therapeutic agent is detectable at a biologically significant level in the blood plasma of the subject.

“Controlled release” refers to the release of the therapeutic agent at such a rate that blood (e.g., plasma) concentrations are maintained within the therapeutic range, but below toxic concentrations over a period of time of about one hour or longer, preferably 12 hours or longer.

“Pharmaceutically acceptable carrier” refers to a pharmaceutically acceptable solvent, suspending agent or vehicle for delivering a drug compound of the present invention to a mammal such as an animal or human.

“Nutraceutical acceptable carrier” refers to a nutraceutical acceptable solvent, suspending agent or vehicle for delivering a compound of the present invention to a mammal such as an animal or human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Nutraceutical acceptable” ingredient, excipient or component is one that is suitable for use with humans and/or animals without undue adverse side effects (such as toxicity, irritation and allergic response) commensurate with a reasonable benefit/risk ratio.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the transitional phase “consisting essentially of,” in one non-limiting aspect, a basic and novel characteristic of the compositions and methods disclosed in this specification includes the compositions' abilities to reduce or prevent influenza and flu like symptoms.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 The development of Visual Analog Scale scores in the HSRx 351 treated group (●) and the placebo group (▪).The data is presented as mean±SD, 0=no problem, 10=pronounced problem. The figure is adapted from Kong 2009.

FIG. 2 Percentage of patients recovered from influenza symptoms in the HSRx 351 and placebo groups after 48 hours of treatment. The figure is adapted from Kong 2009.

FIG. 3 The dose dependent inhibition curve of Influenza A (H1N1) virus infection of MDCK cells incubated with HSRx 351. The IC₅₀ and IC₁₀₀ values were determined using the line of best fit (R²=0.92; n=22). The figure is adapted from Roschek Jr 2009.

FIG. 4 The hemagglutination assay demonstrates that HSRx 351 prevents the binding of influenza virus A to red blood cells. Figure Key: RBC=red blood cells; PBS=phosphate buffered saline; pAB=anti-influenza antibody; virus=influenza virus A; HS932 HSRx 351. The RBCs+PBS row is a negative control row showing the inability of red blood cells to hemagglutinate in PBS alone. The virus+PBS+RBCs row is a negative control row that shows that increasing concentrations of virus from right to left increases hemagglutination of red blood cells (see diffuse red in left 2 wells). The pAb+virus+RBCs row is a positive control row that shows that a constant concentration across the row of an antibody against hemagglutinin inhibits hemagglutination demonstrated by decreased dispersion of red blood cells in the positive control row in comparison to corresponding virus +PBS+RBCs wells, especially in wells two and three from the left. The three HS9+virus+RBCs rows are experimental rows that show that a constant concentration of HSRx 351 (HS9) inhibits hemagglutination similarly to antibody against hemagglutinin.

FIG. 5 The concentration of biomarker 6 when in HSRx 351 and alone required for IC₅₀. The ratio of concentration of biomarker 6 alone to when in HSRx 351 suggests synergistic activity of biomarker 6 with the other components of HSRx 351.

FIG. 6 The concentration of biomarker 7 when in HSRx 351 and the analog alone required for IC₅₀. The ratio of concentration of biomarker 7 analog alone to biomarker 7 in HSRx 351 suggests synergistic activity of biomarker 7 with the other components of HSRx 351.

FIG. 7 The dose dependent inhibition of Zika virus infection of cells incubated with HSRx 351.

FIGS. 8 A and B The bioavailability of biomarkers 6, 7, and 9 in human blood from consumption of a 175 mg oral lozenge (A) and a 350 mg drink (B). Blood serum levels were determined by DART-TOF analysis of blood drawn from human patients for up to four hours post ingestion.

### DETAILED DESCRIPTION

The inventors have surprisingly found that a combination of several compounds that can be found in elderberries can prevent and treat influenza and influenza-like illness and prevent and treat envelope virus infections. The inventors have also found that specific relative concentrations of the compounds act to enhance the ability of the combined compounds to prevent and treat virus infection. In addition, the inventors have found that using compounds of the present invention with additional drugs, such as anti-influenza compounds, enhance the ability of the combined compounds to prevent and treat virus infections. Without wishing to be bound by theory, it is believed that the compounds and compositions disclosed herein are capable of blocking entry of a virus into a cell. Non-limiting examples of influenza viruses include viruses within the Influenzavirus A and Influenzavirus B genus. Non-limiting examples of influenza viruses within these genera include: H1N1, H3N2, H3N5, H5N1, and influenza B virus. Non-limiting examples of viruses that cause influenza-like illness includes rhinovirus. Non-limiting examples of envelope viruses include Zika virus, dengue virus, HIV, and herpes simplex viruses. It is also believed that the compounds and compositions disclosed herein are capable of treating and preventing the symptoms associated with an influenza infection and/or flu like symptoms. Non-limiting examples of symptoms include chills, cough, fatigue, fever, headache, muscle aches, and/or sore throat.

### A. Compounds of the Composition

The composition of the present invention can include one or more of the biomarkers found in Sambucus nigra L. (elderberry) defined by accurate mass of 112.027 amu, 126.032 amu, 155.095 amu, 160.087 amu, 166.099 amu, and 507.342 amu, and combinations thereof. In another embodiment, the composition may further comprise one or more of the biomarkers defined by accurate mass of about 358.146 amu, 478.295 amu, and 606.436 amu found in elderberries and any combinations thereof. Without wishing to be bound by theory it is believed that the biomarkers of the present invention block viral entry into a cell.

In a preferred embodiment, the biomarker or combination of biomarkers has a 90% batch-to-batch chemical consistency of relative abundance for the biomarkers. In another preferred embodiment, the compound or combination of compounds has a 95% and/or 98% batch-to-batch chemical consistency of relative abundance for the biomarkers.

In some aspects of the invention, the compounds of the composition and derivatives and analogues can be made through known synthetic methods. In some aspects of the invention, the compounds of the composition and/or the composition can be synthetically obtained by producing the compound(s) and/or the compositions according to methods known to one of skill in the art in chemical synthesis. In one instance, the compound(s) and/or the compositions are synthesized through organic chemistry methods.

In some aspects of the invention, the compounds of the composition and/or the composition can be isolated from extracts of an organism such as fruits, plants, animals, fungi, bacteria, and/or archaea. Non-limiting examples of fruits include elderberry fruit. The compounds of the composition or the composition can be extracted from the organism using known extraction methods, such as contacting the extract with CO₂, contacting the extract with H₂O, or any combination of EtOH:H₂O, with any method utilizing polymer separating the extract. A non-limiting example of a polymer used for polymer separation includes ADS 5 polymer (Nankai University, China). The extract can include any one of or combination of compounds defined by accurate mass of 112.027 amu, 126.032 amu, 155.095 amu, 160.087 amu, 166.099 amu, and 507.342 amu that are found in elderberries. In one instance the extract can also include one or more of the compounds defined by accurate mass of about 358.146 amu, 478.295 amu, and 606.436 amu found in elderberries and any combination thereof.

In some aspects of the invention, one or more of the compounds of the composition and derivatives and analogues thereof can be made through known synthetic methods known by one of skill in the art and one or more of the compounds of the composition and derivatives and analogues thereof may be isolated from other sources, such as, but not limited to, extracts of fruits and plants.

### B. Actives Defined by DART TOF/MS

The composition of the present invention can include one or more of the compounds defined by accurate mass of about 112.027 amu, 126.032 amu, 155.095 amu, 160.087 amu, 166.099 amu, and 507.342 amu found in elderberries and any combination thereof. The composition of the present invention can further include: one or more of the compounds defined by accurate mass of about 358.146 amu, 478.295 amu, and 606.436 amu found in elderberries and any combination thereof; other products; and/or any combination thereof.

The accurate mass and relative abundances described herein are based on experiments using particular instruments and particular settings and can change from instrument to instrument. There is variability in each measurement. Thus, the accurate mass and relative abundances are defined as being close to as understood by one of ordinary skill in the art. In one non-limiting embodiment the terms are defined to be within 20%, preferably 10%, preferably within 5%, more preferably within 1%, and most preferably within 0.5%. In one non-limiting embodiment, the accurate mass has an error of within +/−20 mmu, preferably 10 mmu, more preferably within 5 mmu, and most preferably within 1 mmu. In one non-limiting embodiment, the relative abundance has an error of +/−20%, preferably 10%, preferably within 5%, and more preferably within 1%, and most preferably within 0.5%.

In a non-limiting example, the compounds of the present invention can be identified using Direct Analysis in Real Time (DART) Time of Flight/Mass Spectrometry (TOF/MS). Specifically, a JEOL DART™ AccuTOF-mass spectrometer from Jeol USA of Peabody, Mass. (JMS-T100LC) can be used. Accurate mass can be determined by subtracting the mass of a proton (1.007825 amu) from the measured mass of the ions produced from the sample. The mass of compounds may be determined in a sample by directly introducing the sample to the ion stream by means of a Dip-IT sampler and a Dip-IT sampler holder (ionSense™). While no sample preparation is required for a simple analysis with the DART, a chemical doped/spiked solution can be used for quantitation relative to a known quantity.

As a non-limiting example, curcumin is not present in elderberry extract and can therefore be used to create a quantitative chemical profile of the bioactive molecules. The settings for the DART ion source can be the following:

Gas: He

Flow: 2.52 LPM@ 50 PSI

Temperature: 250 C

Needle Voltage: 3000V

Grid Electrode Voltage: 250V

Discharge Electrode Voltage: 400V

The settings for the JEOL AccuTOF MS can be the following:

Peaks Voltage: 1000V

Orifice 1 Temperature: 120 C

Detector Voltage: 2600V

Reflectron Voltage: 990.0V

Samples can be analyzed in six replicates by DART-TOF MS. These six replicates can be analyzed to create a single, averaged, filtered, and statistically significant DART fingerprint of the sample. This processed fingerprint can then be used to determine the presence of the bioactive markers by comparison of masses. Due to the initial discovery and identification of these bioactive markers, a simple mass comparison is sufficient to determine their presence in any extract or mixture of chemicals.

All MS have a mass tolerance—a range of acceptable reported masses surrounding the predicted [M+H] or [M−H] value. For the AccuTOF, that mass tolerance is less than 20 millimass units (mmu) (predicted mass +/−10 mmu). Given the same sample and ion source, other TOF-MS may have a higher or lower mass tolerance.

In another non-limiting example, the compounds of the present invention can be determined by DART TOF/MS by using a JEOL DART™ AccuTOF-mass spectrometer from Jeol USA of Peabody, Mass. (JMS-T100LC) executed in the positive ion mode ([M+H]⁺) using the following settings for the DART ion source:

Gas: He

Flow: 3.98 L/min

Needle voltage: 3500 V

Temperature: 300° C.

Electrode 1 Voltage: 150 V

Electrode 2 Voltage: 250 V,

The settings for the JEOL AccuTOF MS can be the following:

Peaks Voltage: 1000V

Orifice 1 Voltage: 20 V

Ring Lens Voltage: 5 V

Orifice 2 Voltage: 5 V

Detector Voltage: 2550V

Calibrations can be performed internally with each sample using a 10% (weight/volume) solution of PEG 600 from Ultra Chemical of North Kingston, R.I. that provided mass markers throughout the required mass range of 100-1000 amu. Calibration tolerances can be held to 5 mmu. Samples can be introduced into the DART He plasma using the closed end of a borosilicate glass melting point capillary tube until a signal is achieved in the total-ion chromatogram (TIC). The next sample can then be introduced when the TIC returned baseline levels.

### C. Additional Anti-Viral Drugs

Anti-viral drugs can, but are not limited to, inhibit viral entry into a host cell, prevent budding of virus from a host cell, prevent replication in a host cell, or destroy or inhibit the virus particle. Anti-viral drugs include those that are specific to one or a few viruses or are broad spectrum against several types of viruses. Anti-viral drugs include those that are combination drugs and single drugs. Anti-influenza drugs are a non-limiting example of anti-viral drugs. In one embodiment, the compositions disclosed herein further includes at least one additional anti-viral drug.

Anti-influenza agents are compounds or compositions that are used to decrease the influenza viral load or prevent viral infection. Non-limiting examples of anti-influenza agents include oseltamivir (also known as TAMIFLU®), zanamivir (RELENZA®), peramivir (RAPIVAB®) rimantadine (also known as FLUMADINE®), and amantadine (also known as SYMMETREL®). Some anti-influenza agents inhibit neuraminidase, which prevents the release of viral progeny from infected cells. Non-limiting examples of anti-influenza agents that prevent the release of viral progeny from infected cells include neuraminidase inhibitors such as oseltamivir, zanamivir, and peramivir. Some anti-influenza agents block the viral encoded M2 ion-channel. Non-limiting examples of anti-influenza agents that block the M2 ion-channel are rimantadine and amantadine. Non-limiting examples of influenza viruses include viruses of the Influenzavirus A and Influenzavirus B genus. In one instance the viruses include, but are not limited to, H1N1, H3N2, H3N5, H5N1, and Influenza B. In one embodiment, the compositions disclosed herein further includes at least one additional anti-influenza agent, which may be, but is not limited to, oseltamivir, zanamivir, peramivir, rimantadine, and amantadine.

### D. Amounts of Ingredients

It is contemplated that the compositions of the present invention can include any amount of the ingredients discussed in this specification. The compositions can also include any number of combinations of additional ingredients described throughout this specification (e.g., stabilizers, fillers, pharmaceutically and/or nutraceutical acceptable salts, and/or additional pharmaceutical and/or nutraceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition or relative abundance. A person of ordinary skill in the art would understand that the concentrations can vary depending on the addition, substitution, and/or subtraction of ingredients in a given composition.

### E. Additional Components

The compound of the present invention can be formulated into any suitable composition form for administration to a human or non-human animal patient.

The composition may consist of the claimed compounds alone or may include the compounds and any suitable additional component, such as one or more pharmaceutically and/or nutraceutical acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient.

1. Excipients

Excipients employed in the compositions of the present invention can be solids, semi-solids, liquids or combinations thereof. Preferably, the excipients are solids. Compositions of the invention containing excipients can be prepared by any known technique that comprises, for example, admixing an excipient with the claimed compounds. A pharmaceutical composition of the invention contains a desired amount of the claimed compounds per dose unit and, if intended for oral administration, can be in the form, for example, of a tablet, a caplet, a pill, a hard or soft capsule, a lozenge, a cachet, a dispensable powder, granules, a suspension, an elixir, a dispersion, or any other form reasonably adapted for such administration. If intended for parenteral administration, it can be in the form, for example, of a suspension or transdermal patch. If intended for rectal administration, it can be in the form, for example, of a suppository. Presently preferred are oral dosage forms that are discrete dose units each containing a predetermined amount of the claimed compounds such as tablets or capsules.

2. Carriers/Diluents

Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

3. Disintegrant

Compositions of the invention optionally can include one or more pharmaceutically and/or nutraceutical acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

4. Binders

The compositions of the present invention can include binding agents or adhesives particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, HPMC, hydroxypropylcellulose, and ethylcellulose. Such binding agents and/or adhesives, if present, constitute in total about 0.5% to about 25%, preferably about 0.75% to about 15%, and more preferably about 1% to about 10%, of the total weight of the pharmaceutical composition. Many of the binding agents are polymers comprising amide, ester, ether, alcohol or ketone groups and, as such, can be included in pharmaceutical compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer affects properties and performance.

5. Wetting Agents

Wetting agents can be used in the compositions of the present invention. Wetting agent can be selected to maintain the crystal in close association with water, a condition that may improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the pharmaceutical composition.

6. Lubricants

Lubricants can be included in the compositions of the present invention. Suitable lubricants include, but are not limited to, either individually or in combination, glyceryl behapate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, constitute in total about 0.1% to about 10%, preferably about 0.2% to about 8%, and more preferably about 0.25% to about 5%, of the total weight of the composition.

7. Other Agents

Surfactant, emulsifier, or effervescent agents can be used in the compositions. Emulsifying agents can be used to help solubilize the ingredients within a soft gelatin capsule. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

### F. Vehicles

Various delivery systems are known in the art and can be used to administer a therapeutic agent or composition of the invention, e.g., encapsulation in liposomes, microparticles, microcapsules, receptor-mediated endocytosis and the like. Methods of administration include, but are not limited to, parenteral, intra-arterial, intramuscular, intravenous, intranasal, and oral routes. The compositions can be provided in the form of tablets, lozenges, granules, capsules, pills, ampoule, suppositories or aerosol form. The compositions can also be provided in the form of suspensions, solutions, and emulsions of the active ingredient in aqueous or non-aqueous diluents, syrups, granulates or powders.

### G. Formulation and Administration

The composition may, for example, be a pharmaceutical composition (medicament), and over the counter composition (OTC), a nutraceutical, etc. Compositions according to the present invention include formulations suitable for oral or parenteral routes. Non-limiting examples of specific routes include intradermal, subcutaneous, intramuscular, intravenous, local injection, rectal, intranasal inhalation, insufflation, topical (including transdermal, buccal and sublingual), vaginal, parenteral (including subcutaneous, intramuscular, intravenous and intradermal) and pulmonary administration. The formulations can conveniently be presented in unit dosage form and can be prepared by any methods well known in the art. Such methods include the step of bringing into association the active ingredient (or ingredients) with the carrier, which constitutes one or more accessory ingredients. In general, the formulations are prepared by uniformly and intimately bringing into association the active ingredient with a suitable carrier, such as liquid carriers or finely divided solid carriers or both, and then if necessary shaping the product. Formulations of the subject invention suitable for oral administration can be presented as discrete units such as capsules, cachets or tablets, each containing a predetermined amount of the active ingredient, or as an oil-in-water liquid emulsion, water-in-oil liquid emulsion, or as a supplement within an aqueous solution, for example, a tea. The active ingredient can also be presented as bolus, electuary, or paste. Useful injectable preparations include sterile suspensions, solutions or emulsions of the compound compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives. Alternatively, the injectable formulation can be provided in powder form for reconstitution with a suitable vehicle, including but not limited to sterile pyrogen free water, buffer, dextrose solution, etc., before use. To this end, the compound compositions can be dried by any art-known technique, such as lyophilization, and reconstituted prior to use.

Formulations suitable for topical administration in the mouth include lozenges comprising the active ingredient in a flavored basis, usually sucrose and acacia or tragacanth, pastilles that include the active ingredient in an inert basis such as gelatin and glycerin, or sucrose and acacia, mouthwashes that include the active ingredient in a suitable liquid carrier, and chocolate comprising the active ingredients.

Formulations suitable for topical administration according to the subject invention can be formulated as an ointment, cream, suspension, lotion, powder, solution, paste, gel, spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for intranasal administration, such as by the non-limiting examples of a nebulizer, include aqueous or oily solutions of the agent. Formulations preferably can include compounds that facilitate absorption of the active ingredients through the skin and into the bloodstream.

Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents, and liposomes or other microparticulate systems which are designed to target the compound to blood components or one or more organs. The formulations can be presented in unit-dose or multi-dose or multi-dose sealed containers, such as for example, ampoules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically and/or nutraceutical acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

For buccal administration, the compositions can take the form of the non-limiting examples of tablets or lozenges formulated in a conventional manner.

For rectal and vaginal routes of administration, the compound compositions can be formulated as solutions (for retention enemas) suppositories or ointments containing conventional suppository bases such as cocoa butter or other glycerides.

For nasal administration or administration by inhalation or insufflation, the compound compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the compound compositions can be formulated as a depot preparation for administration by implantation or intramuscular injection. The compound compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compound compositions for percutaneous absorption, can be used. To this end, permeation enhancers can be used to facilitate transdermal penetration of the compound compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver the compound compositions. Certain organic solvents such as dimethylsulfoxide (DMSO) can also be employed, although usually at the cost of greater toxicity.

It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

In one embodiment, the pharmaceutical and/or nutraceutical compositions of the invention can be administered locally to the area in need of treatment; such local administration can be achieved, for example, by local infusion, by injection, or by means of a catheter. In another embodiment, a compound or composition of the invention is administered in a manner so as to achieve peak concentrations of the active compound at sites of the disease. Peak concentrations at disease sites can be achieved, for example, by intravenously injecting of the agent, optionally in saline, or orally administering, for example, a tablet, capsule or syrup containing the active ingredient.

H. Other Pharmaceutical and/or Nutraceutical Agents

Pharmaceutical, OTC, and/or nutraceutical formulations of the invention can be administered simultaneously or sequentially with other drugs or biologically active agents. Examples include, but are not limited to, anti-influenza agents, antioxidants, free radical scavenging agents, analgesics, anesthetics, anorectals, antihistamines, anti-inflammatory agents including non-steroidal anti-inflammatory drugs, antibiotics, antifungals, antivirals, antimicrobials, anti-cancer actives, antineoplastics, biologically active proteins and peptides, enzymes, hemostatics, steroids including hormones and corticosteroids, etc.

### I. Therapeutic Methods And Dosage

Preferred unit dosage formulations are those containing a daily dose or unit, daily subdose, or an appropriate fraction thereof, of an agent. Therapeutic amounts can be empirically determined and will vary with the pathology being treated, the subject being treated, and the efficacy and toxicity of the agent. Similarly, suitable dosage formulations and methods of administering the agents can be readily determined by those of ordinary skill in the art.

In some embodiments, a therapeutic method of the present invention can include treating a disease, condition, or disorder by administering to a subject having such disease or condition a stable formulation as described herein in an amount effective to treat the disease, condition, or disorder. In some embodiments, the subject is administered a stable formulation comprising the compounds claimed herein. The disease, condition, or disorder can be caused by an influenza virus. Further, the disease, condition, or disorder can be influenza, the flu, and/or a disease with flu like symptoms and related diseases, conditions, and disorders. For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art. In some aspects of the invention, total dosage amounts of a compound composition will typically be in the range of from about 0.0001 or 0.001 or 0.01 mg/kg of patient/day to about 100 mg/kg patient/day, but may be higher or lower, depending upon, among other factors, the activity of the components, its bioavailability, the mode of administration and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated and the judgment of the prescribing physician. In another non-limiting example, the compounds can be administered to a subject for 1 day, 2 days, 3 days, 4 days, 5 days, 6 days, a week, or more. Skilled artisans will be able to optimize effective local dosages without undue experimentation.

### J. Kits

In another aspect of the present invention, kits for treating a disease, condition or disorder as described herein. For instance, compositions of the present invention can be included in a kit. A kit can include a container. Containers can include a bottle, a metal tube, a laminate tube, a plastic tube, a dispenser, a straw, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a predetermined amount of the composition. In other embodiments, the container can be squeezed (e.g., metal, laminate, or plastic tube) to dispense a desired amount of the composition. The composition can be dispensed as a spray, an aerosol, a liquid, a fluid, a semi-solid, or a solid. In a preferred embodiment, the composition is dispensed as a tablet or lozenge. The containers can have spray, pump, or squeeze mechanisms. A kit can also include instructions for employing the kit components as well the use of any other compositions included in the container. Instructions can include an explanation of how to apply, use, and maintain the compositions. The compositions can, if desired, be presented in a pack or dispenser device, which can contain one or more unit dosage forms containing the compound compositions. The pack can, for example, comprise metal or plastic foil, such as a blister pack. The pack or dispenser device can be accompanied by instructions for administration.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

The results, in combination, surprisingly show that the compositions disclosed herein can be used to treat and prevent influenza and influenza-like illnesses and can be used to treat and prevent envelope virus infection.

### Example 1

### Characterization of Compounds by Accurate Mass and Relative Abundance)

The inventors have surprisingly found that a combination of several compounds found in elderberries can prevent and treat influenza virus infection. The inventors have also found that specific relative concentrations of the compounds act to enhance the ability of the combined compounds to prevent and treat influenza virus infection. In addition, the inventors have found that using compounds of the present invention with additional anti-influenza compounds also enhance the ability of the combined compounds to prevent and treat influenza virus infection. The compounds of the present invention include biomarker compounds defined by compounds found in Sambucus nigra with an accurate mass of 112.027 amu, 126.032 amu, 155.095 amu, 160.087 amu, 166.099 amu, and 507.342 amu. These compounds may be produced synthetically or isolated from an organism such as, but not limited to, Sambucus nigra. The composition may further contain biomarker compounds found in Sambucus nigra with an accurate mass of 358.146 amu, 478.295 amu, and 606.436 amu. The compounds may be characterized by methods known by one of skill in the art.

Accurate mass and relative abundances described herein are based on experiments using particular instruments and particular settings and can change from instrument to instrument. There is variability in each measurement. Thus, the accurate mass and relative abundances are defined as being close to as understood by one of ordinary skill in the art. In one non-limiting embodiment the terms are defined to be within 20%, preferably 10%, preferably within 5%, more preferably within 1%, and most preferably within 0.5%. In one non-limiting embodiment, the accurate mass has an error of within +/−20 mmu, preferably 10 mmu, more preferably within 5 mmu, and most preferably within 1 mmu. In one non-limiting embodiment, the relative abundance has an error of +/−20%, preferably 10%, preferably within 5%, and more preferably within 1%, and most preferably within 0.5%.

Methods for Accurate mass: The compounds were characterized and relative abundance was determined using Direct Analysis in Real Time (DART) ion source combined with Time of Flight/Mass Spectrometry (TOF-MS). Specifically, the DART TOF-MS was a JEOL DART™ AccuTOF-mass spectrometer from Jeol USA of Peabody, Mass. (JMS-T100LC). The mass of the compounds were determined in a Sambucus nigra extract sample by directly introducing the sample to the ion stream by means of a Dip-IT sampler and a Dip-IT sampler holder (ionSense™).

The settings for the DART ion source were the following:

Gas: He

Flow: 2.52 LPM @ 50 PSI

Temperature: 250 C

Needle Voltage: 3000V

Grid Electrode Voltage: 250V

Discharge Electrode Voltage: 400V

The settings for the JEOL AccuTOF MS were the following:

Peaks Voltage: 1000V

Orifice 1 Temperature: 120 C

Detector Voltage: 2600V

Reflectron Voltage: 990.0V

Extract samples were analyzed in six replicates by DART-TOF MS. These six replicates were analyzed to create a single, averaged, filtered, and statistically significant DART fingerprint of the extract. This processed fingerprint was then used to determine the presence of the bioactive markers by comparison of masses. Due to the initial discovery and identification of these bioactive markers, a simple mass comparison was sufficient to determine their presence in any extract or mixture of chemicals. For the AccuTOF, that mass tolerance is less than 20 millimass units (mmu) (predicted mass +/−10 mmu). Given the same extract and ion source, other TOF mass spectrometers may have a higher or lower mass tolerance.

Methods for Relative Abundance: While no sample preparation is required for a simple analysis with the DART, a curcumin doped/spiked solution was used for determining relative abundance of test compositions through quantitation relative to a known quantity. Standards that are well known and that exist naturally in elderberry, such as rutin, would vary given any number of influences—growing conditions, harvest time, plant health, etc. For purposes of quantifying the biomarkers, the natural variations of rutin (or other naturally occurring standards) make it unacceptable to use as a basis for an absolute quantification of the biomarkers. In order to remove that inconsistency, a compound that is not native to elderberry (in this case, curcumin) was used as the basis for a quantitative chemical profile of the bioactive molecules.

For determining relative abundance of samples with unknown concentrations of the biomarkers disclosed herein, 1 mg/ml samples of the disclosed compositions were doped/spiked with 0.01 mg/ml curcumin. Samples were then analyzed by the DART-TOF method used above.

Table 1 discloses the relative abundance of the biomarkers disclosed herein found in non-limiting, preferred embodiments of compositions comprising all nine biomarkers.

TABLE 1
Relative Abundance of the biomarkers in preferred
active compositions determined using 1 mg/ml of the
compositions spiked with 0.01 mg/ml curcumin.

| | Minimum Relative | Maximum Relative |
|---|---|---|
| | Concentration | Concentration |
| | (−20%) | (+20%) |
| Biomarker 1 | 2.36% | 6.94% |
| Biomarker 2 | 33.26% | 85.75% |
| Biomarker 3 | 1.86% | 4.69% |
| Biomarker 4 | 5.03% | 12.89% |
| Biomarker 5 | 9.26% | 24.11% |
| Biomarker 6 | 11.37% | 31.81% |
| Biomarker 7 | 1.20% | 3.40% |
| Biomarker 8 | 0.60% | 1.75% |
| Biomarker 9 | 0.07% | 1.38% |

### Example 2

### Formulations for Examples 3 through 8)

A dose-reliable, elderberry extract comprising biomarkers 1 through 9 with in vitro and in vivo anti-viral activity was produced in general according to the methods described in Fink et al. 2009 and Roschek Jr. et al. 2009.

Generally, elderberry fruits (Sambucus nigra L.) were ground and extracted with EtOH:H₂O (4:1, v/v). The collected fraction was dried at 50° C. overnight to yield a crystalline powder. The procedure was repeated multiple times to ensure reproducibility of the extract.

### Example 3

### Safety

HSRx 351 was tested to determine cellular toxicity in vitro. It was determined that HSRx 351 shows no signs of toxicity using a standard mitochondrial reductase activity assay (MTT). The MTT assay measured cell metabolism in MDCK cells. No toxicity was shown at any concentration tested (from 0.02 mg/ml to 2.4 mg/ml).

### Example 4

### Bioavailability

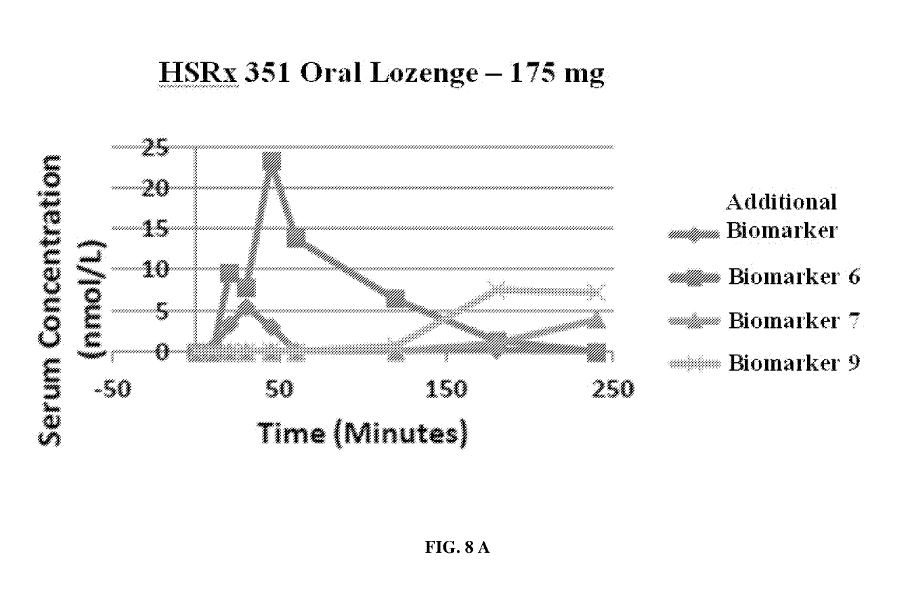

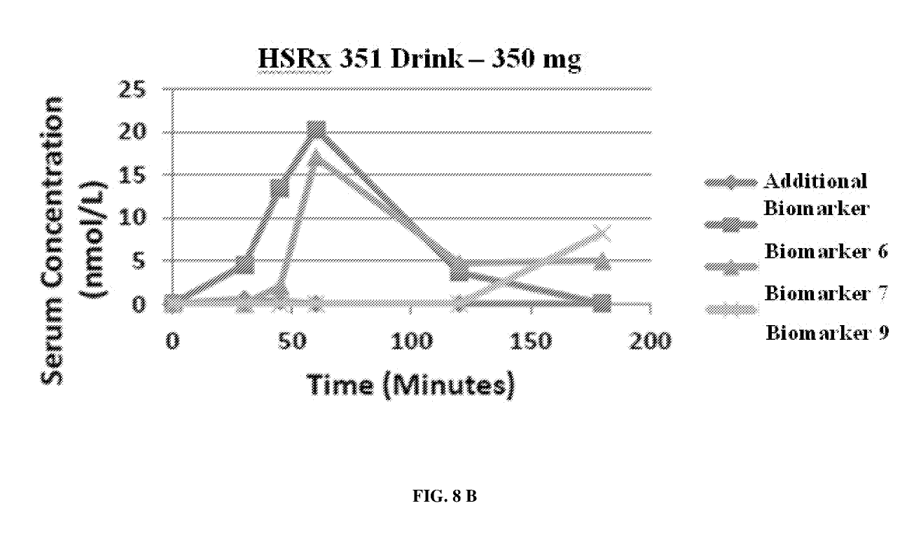

HSRx 351 was tested to determine bioavailability in human subjects using an oral lozenge containing 175 mg of HSRx 351 and a drink containing 350 mg of HSRx 351. It was determined that biomarkers were seen in the blood of human subjects as early as 20 minutes after consumption, while some remained in the blood stream for over 4 hours. The procedures and results are described in Roschek and Alberte 2008 and the results are shown below in Table 2, FIG. 8 A, and FIG. 8 B.

Lozenge—Briefly, six subjects were placed on a diet free of flavonoids for 24 hours prior to the initiation of the study. Blood samples were collected at several time intervals between 0 and 480 minutes. The subjects were given a lozenge containing 175 mg of HSRx 351 immediately after time zero and allowed the lozenge to dissolve slowly in the oral cavity.

Drink—Briefly, one subject fasted for 24 hours prior to the initiation of blood collection and consumption of the drink. During the course of the study, the subject only received water and food absent flavonoids. A blood sample was collected at several time intervals between 0 and 360 minutes. The subject was given an 8 ounce drink that contained dissolved therein two lozenges that contained a total of 350 mg of HSRx 351 immediately after time zero.

TABLE 2
Percent bioavailability of biomarkers 6, 7, and 9 in HSRx 351 delivered
orally by dissolving a lozenge in the oral cavity or by ingesting
dissolved lozenges (adapted from Roschek and Alberte 2008).

% Bioavailability in Blood Serum

| | Lozenge | Drink |
|---|---|---|
| Biomarker 6 | 10.5 | 4.6 |
| Biomarker 7 | 8.6 | 18.8 |
| Biomarker 9 | 19.7 | 10.8 |

### Example 5

### Treatment of Influenza Infection and/or Influenza-Like Symptoms

HSRx 351, a preferred embodiment of the disclosed composition that comprises biomarkers 1 through 9, was tested to determine the anti-viral properties of the composition and the efficacy in relieving symptoms of influenza in human subjects.

Human Study—For the human study, the ability of HSRx 351 to treat six flu and/or flu like symptoms were evaluated. The study showed that HSRx 351 reduces all six symptoms. The methodology used is described in Kong 2009.

Treatment: Briefly, the HSRx 351 composition was formulated as a slow-dissolve lozenge containing 175 mg total of biomarkers 1 through 9. A placebo lozenge, identical in appearance, taste, and composition except for it lacked HSRx 351 was supplied in similar packaging. A randomized, double-blind, placebo-controlled pilot clinical trial was conducted to evaluate the efficacy of the test composition for treatment of flu and/or flu-like symptoms. 64 volunteers (age ranged 16 to 60 years) presenting flu symptoms for less than 24 hours but otherwise healthy were included in the study. The participants had at least three of the following symptoms: fever, headache, muscle aches, coughing, mucus discharge, and nasal congestion. Patients who were pregnant, breastfeeding, suffered from chronic diseases, were suspected of having a bacterial infection, participated in another clinical trial, or recently received flu medication, antiviral therapy, or influenza vaccination were excluded from the study. Patients were asked to take four of either the HSRx 351 lozenge (n=32) or placebo lozenges (n=32) a day for two days, one before each meal and one before bed. The first dose of medication was administered immediately after the investigator made the decision to enroll the patient into the study.

Evaluation: The severity of six flu-like symptoms were assessed to determine the efficacy of HSRx 351: fever, headache, muscle aches, coughing, mucus discharge, and nasal congestion. To determine if the placebo and treatment group were clinically comparable, the patients symptoms were assessed at the onset of treatment (baseline) on the Visual Analogue Scales (VAS) from 0=no problems to 10=pronounced problems. Thereafter, patients were instructed to self-evaluate their symptoms by VAS and score their symptom improvements four times a day after administration of the lozenge during the two day treatment. The assessments were used for statistical analysis.

Statistical analysis: Variables assumed to be continuous were expressed as mean values, with 95% confidence intervals constructed using Student's t-distribution method. The standard deviation and total ranges were used as indices of distribution. Both inter- and intra-group analyses were carried out using two-tailed tests with a significance level of 5%. The continuously distributed variables were analyzed using the analysis of variance model with repeated measurements in order to compare both between and within groups.

Results: No obvious differences were observed in demographic characteristics between the placebo group and HSRx 351 treatment group (Table 3). Before the first treatment, the patient's flu-like symptoms were evaluated (Table 4). The mean VAS scores of most symptoms before the first treatment showed no significant differences between the two groups (p>0.05) except for the mean VAS score for fever (p=0.0256) (Table 5A)

TABLE 3
Demographic characteristics of the included
patients (adapted from Kong 2009).

| | |
|---|---|
| HSRx 351 Treatment | Placebo |
| Group | Group |

| | | |
|---|---|---|
| Total number | 32 | 32 |
| Male | 17 | 17 |
| Female | 15 | 15 |

| | | | | |
|---|---|---|---|---|
| Age range | 20-55 | years | 27-59 | years |
| Mean age | 40 | years | 40.1 | years |

TABLE 4
Distribution of symptoms in treatment and control groups
at the beginning of the study (adapted from Kong 2009).

| | HSRx 351 Treatment | Placebo |
|---|---|---|
| Symptoms | Group % | Group % |
| Head ache | 100 | 100 |
| Nasal congestion | 100 | 87.5 |
| Muscle aches | 96.9 | 93.8 |
| Coughing | 50 | 50 |
| Mucus discharge | 50 | 34.3 |
| Fever | 46.9 | 28 |

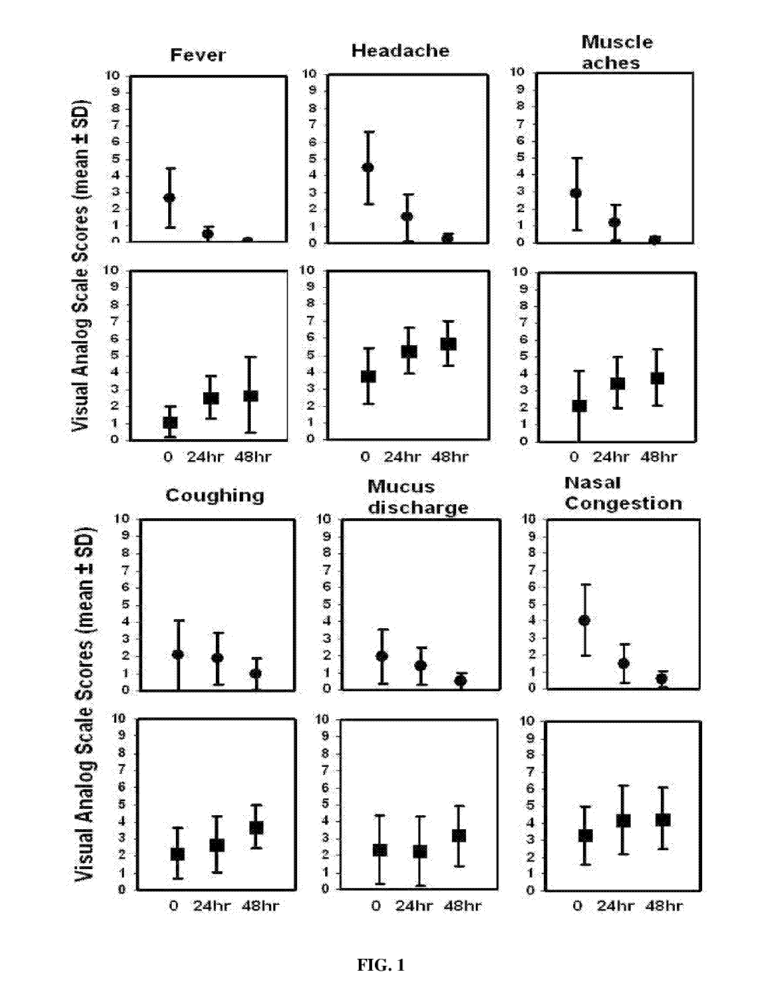

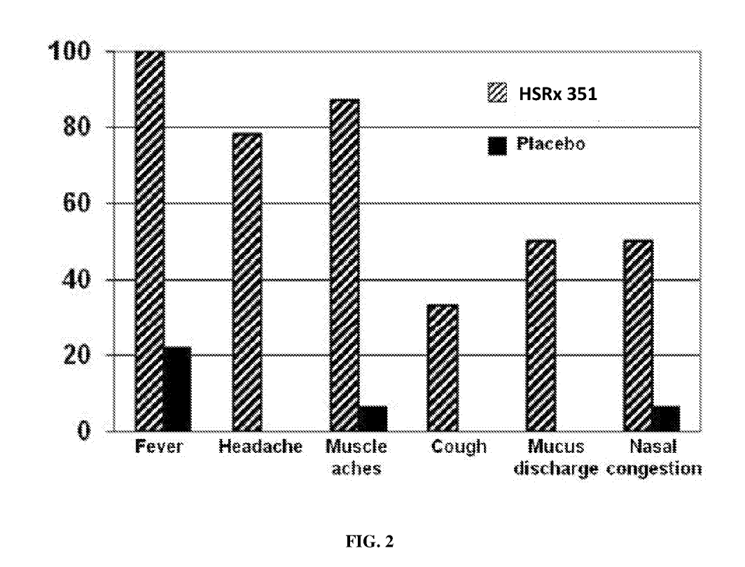

Fever: 15 out of 32 (46.9%) patients in the HSRx 351 group and 9 out of 32 (28.1%) patients in the placebo group had fever at the onset of the study (Table 4). The temperatures ranged between 37.3 to 38.8° C. Following the first 24 hours of treatment, the HSRx 351 group showed significant reduction in fever as evidenced by a decrease in the mean VAS score from 2.67±1.80 to 0.47±0.64 (p<0.0001) (FIGS. 1) and 60% of the fever patients returned to normal temperature (FIG. 2). All patients with fever in the HSRx 351 group returned to normal temperature within 48 hours (FIG. 2). In the placebo group, the majority of the patients failed to show any improvement in fever within the 48-hour treatment period, and only 2 patients (22%) in this group returned to normal temperature (FIG. 2).

Headache: All patients in both groups reported headaches at the onset of the study (Table 4). Through 24 hours of treatment, the HSRx 351 group showed a significant reduction in headache symptom. The mean VAS score decreased from 4.47±2.14 to 1.53±1.41 (p<0.0001) (FIG. 1). By 48 hours, the mean VAS score for the HSRx 351 group was close to zero (0.28±0.63) (FIGS. 1) and 78% of patients in this group were free of headaches (FIG. 2) while the remaining 22% reported only mild headaches (VAS=1). In contrast, headaches became more severe in the placebo group where the mean VAS score increased from 3.78±1.66 to 5.25±1.34 (p<0.0001) over the 48-hour treatment period (FIG. 1). No improvement in headache was reported by any single individual subject in the placebo group.

Muscle aches: Over 90% of the patients in both groups reported muscle aches (Table 4). The mean VAS score in the HSRx 351 group decreased from 2.87±2.13 to 1.19±1.05 (p=0.0002) within 24 hours (FIG. 1), indicating a significant improvement in symptoms. By 48 hours, 87% of the patients had completely recovered from muscle aches (FIG. 2), and the mean VAS score reached 0.16±0.45 (FIG. 1). The placebo group reported a worsening of muscle aches as the mean VAS score increased from 2.13±2.10 to 3.47±1.50 (p=0.0013) at 48 hours (FIG. 1).

Nasal congestion: All patients in the HSRx 351 group and 87.5% of patients in the placebo group reported nasal congestion when enrolled in the study (Table 4). By 24 hours into the treatment, the HSRx 351 group showed significant improvement in symptoms. The mean VAS score for this group decreased from 4.03±2.10 to 1.47±1.14 (p<0.0001) (FIG. 1). By 48 hours, the mean VAS score dropped to 0.56±0.62 (FIGS. 1) and 50% of the patients were symptom free (FIG. 2). In contrast, nasal congestion in the placebo group worsened in most individuals at 48 hours. The mean VAS score in this group increased from 3.30±1.71 to 4.26±1.81 (p=0.049) (FIG. 1). Only 2 out of 30 patients (7%) in the placebo group showed alleviation of nasal congestion.

Nasal mucus discharge: Nasal mucus discharge was a less common and less severe symptom among patients in both study groups. Only 50% of patients in the HSRx 351 group and 34.3% of patients in the placebo group reported nasal mucus discharge (Table 4). Although patients in the HSRx 351 group showed some improvement over the 24-hour treatment (FIG. 1), the improvement was not significant (p=0.26). By 48 hours of treatment, the HSRx 351 group showed significant symptom improvement with the mean VAS score decreasing from 1.94±1.61 to 0.50±0.52 (p=0.0019) (FIG. 1), 8 out of 16 patients (50%) reporting no symptoms and the remaining 50% reporting only mild symptoms (VAS=1). In the placebo group, only 1 out 16 (6%) reported symptom improvement, whereas the remaining 15 patients showed no symptom improvement.

Coughing: Fifty percent of the patients in both groups reported coughing when enrolled into the study (Table 4). In the HSRx 351 group, coughing persisted longer than the other symptoms. No significant improvement was recorded for this group over the 24-hour treatment period (FIG. 1). By 48 hours, 5 out of 16 patients (31%) were relieved from coughing and 6 patients (37%) showed symptom improvement (VAS=1). Although the mean VAS score decreased from 2.07±2.19 to 1.00±0.926 (FIG. 1), this decrease was not statistically significant (p=0.093). However, intergroup comparison (Table 5C) revealed that coughing was also significantly improved (p<0.0001) in the HSRx 351 group. In the placebo group, 14 out of 16 (87%) patients showed worsening symptoms and the remaining 2 patients (13%) showed slight symptom improvement. The mean VAS score in the placebo group increased from 2.19±1.47 to 3.69±1.25 (p=0.0041) (FIG. 1).

Adverse effects: No adverse reactions related to the treatment were reported by either group.

Results: The results show that HSRx 351 can rapidly relieve flu-like symptoms. The HSRx 351 group showed significant improvement of symptoms within 24 hours of the onset of treatment, while the placebo group so no symptom improvement. Within 24 hours, systemic (fever, headache, and muscle aches) and nasal symptoms (nasal congestion) were all significantly reduced in the HSRx 351 treatment group. Cough and nasal mucus discharge did not show significant improvement at 24 hours, but did show improvement within 48 hours of treatment. At 48 hours of treatment, nearly 90% of the

HSRx 351 treated patients were either symptom free or had only mild symptoms (VAS=1). Previously, elderberry syrup was shown to reduce the duration of flu symptoms by 3-4 days (Zakay-Rones et al. 1995; Zakay-Rones et al. 2004). In comparison, a reduction of only 2-2.5 days was reported for the neuraminidase inhibitor drugs oseltamivir and zanamivir treatment (Monto et al. 1999; Makela et al. 2000; Nicholson et al. 2000). These results surprisingly show that HSRx 351 has similar or even superior efficacy than the currently used anti-viral drugs or elderberry syrup in improving symptoms and shortening the duration of influenza.

In the clinical study here, the HSRx 351 was shown to be safe as no patients receiving the HSRx 351 reported any adverse events including nausea and vomiting, which are two adverse-events common in anti-viral treatments (Nicholson et al. 2000).

TABLE 5
Comparison of the VAS Scores of HSRx 351 Treatment Group
and placebo treated groups at the onset (A), 24 hours (B)
and 48 hours (C) of treatment (adapted from Kong 2009).

| | | | |
|---|---|---|---|
| | HSRx 351 Treatment | | |
| | Group | Placebo Group | |
| Symptoms | mean ± SD | mean ± SD | p value |

A.

| | | | |
|---|---|---|---|
| Headache | 4.47 ± 2.14 | 3.78 ± 1.66 | 0.1561 |
| Nasal congestion | 4.03 ± 2.10 | 3.30 ± 1.71 | 0.1508 |
| Muscle aches | 2.87 ± 2.13 | 2.13 ± 2.10 | 0.1777 |
| Coughing | 2.07 ± 2.19 | 2.19 ± 1.47 | 0.8571 |
| Mucus discharge | 1.94 ± 1.61 | 2.36 ± 2.01 | 0.5473 |
| Fever | 2.67 ± 1.80 | 1.11 ± 0.93 | 0.0256 |

B.

| | | | |
|---|---|---|---|
| Headache | 1.53 ± 1.41 | 5.25 ± 1.34 | <0.0001 |
| Nasal congestion | 1.47 ± 1.14 | 4.19 ± 2.02 | <0.0001 |
| Muscle aches | 1.19 ± 0.77 | 3.47 ± 1.50 | <0.0001 |
| Coughing | 1.87 ± 1.07 | 2.69 ± 1.62 | 0.1556 |
| Mucus discharge | 1.38 ± 1.09 | 2.27 ± 2.05 | 0.1513 |
| Fever | 0.48 ± 0.64 | 2.56 ± 1.24 | <0.0001 |

C.

| | | | |
|---|---|---|---|
| Headache | 0.28 ± 0.63 | 5.69 ± 1.35 | <0.0001 |
| Nasal congestion | 0.56 ± 0.62 | 4.26 ± 1.81 | <0.0001 |
| Muscle aches | 0.16 ± 0.45 | 3.80 ± 1.69 | <0.0001 |
| Coughing | 1.00 ± 0.92 | 3.69 ± 1.25 | <0.0001 |
| Mucus discharge | 0.50 ± 0.52 | 3.18 ± 1.78 | <0.0001 |
| Fever | 0 | 2.67 ± 2.24 | <0.0001 |

A score of 0 indicates no problems, and a score of 10 indicates pronounced problem.

### Example 6

### Treatment of Influenza Infection and/or Influenza-Like Symptoms in Combination with Tamiflu®

Otherwise healthy subjects with influenza symptoms were enrolled to evaluate the effect of the HSRx 351 on improving signs and symptoms of influenza or influenza-like illness when combined with Tamiflu®. The study was a randomized, single-blind, placebo controlled, comparative therapeutic clinical study and assessment.

Methodology: 58 Subjects who meet all of the inclusion/exclusion criteria and who presented symptoms consistent with influenza or influenza-like illness were enrolled in the treatment groups D or C. Exclusion criteria included subjects less than 16 years of age and over 70 years of age, or individuals who were pregnant, breastfeeding, suffered from chronic diseases, were suspected of having a bacterial infection, participated in another clinical trial, or recently received flu medication, antiviral therapy, or influenza vaccination.

Subjects were randomized and placed on a treatment regimen of five days. Group C contained 29 subjects that received 75 mg of Tamiflu® (Oseltamivir Phosphate) in capsule form two times a day for five days. Group D contained 29 subjects that received 75 mg of Tamiflu® (Oseltamivir Phosphate) in capsule form and two 175 mg lozenges of HSRx 351 two times a day for 5 days. After a screening visit, subjects returned for visits on day 3, 5, and 10.

The efficacy of the treatment was evaluated based on the study investigator assessing symptoms and overall well-being. The symptoms assessed were: 1) aches and pains; 2)degree of coughing; 3) frequency of coughing; 4) quality of sleep; 5) mucus discharge in the respiratory tract; 6) nasal congestion; and 7) fever reduction. Symptoms were assessed at the baseline visit to determine if the two groups were clinically comparable at the start of the study. The subjects scored their symptoms on the Visual Analogue Scales (VAS) from 0=no problems to 10=pronounced problems, at the baseline visit and then four times a day during the 5 day treatment and two times a day for five days after the treatment has finished. Subjects were also instructed to record any adverse events. On return visits, study personnel marked their assessment of the subject using the same scale.

Statistical Analysis/Results—A survival analysis was performed to compare over time the Influenza symptom functions of groups C and D. The time dependent event for which survival and hazard functions were calculated was the final data collection visit for each subject. A Generalized Linear Mixed Model was run to test the hypothesis of fewer adverse events in the combination treatment Group D versus Tamiflu® (Oseltamivir phosphate) only treatment groups C. Parameter estimates such as beta weights and R2 were evaluated along with a P value significance level set at 5%.

Results: The additional use of HSRx 351 increased the rate at which subjects recover from influenza and influenza-like illness in all parameters except for reduction in the degree of coughing, where both groups showed no significant differences (both were within the margin of error). See Table 6B. The combination treatment also decreased symptoms of the flu and influenza-like illness and decreased the adverse side effects from the use of Tamiflu®. See Table 6A and 6B.

TABLE 6A
Preliminary results for the clinical study of treatment
of Influenza infection and/or Influenza-like symptoms
in combination with Tamiflu ®

| | HSRx 351 + Tamiflu ® | Tamiflu ® Alone |
|---|---|---|
| | (number of subjects) | (number of subjects) |
| | Composite VAS | Composite VAS |
| | assessment exceeding | assessment exceeding |
| | baseline VAS = 65 | baseline VAS = 126 |
| At least one symptom | 2 | 6 |
| worsened by end of | | |
| study | | |
| Multiple symptoms | 0 | 3 |
| worsened by end of | | |
| study | | |
| Symptoms failed to | 0 | 2 |
| improve by end of | | |
| study | | |
| Adverse events | 1 | 4 |
| attributed to study | | |
| product | | |

TABLE 6B
Preliminary statistical results for the clinical study
of treatment of Influenza infection and/or Influenza-
like symptoms in combination with Tamiflu ®

| | | | % Improvement |
|---|---|---|---|
| | HSRx 351 + | Tamiflu ® | Efficacy of |
| | Tamiflu ® | Alone | Combination |
| | (% Average | (% Average | Over Tamiflu ® |
| | Reduction) | Reduction) | Alone |
| Aches and pains | 70.01 | 64.46 | 8.60 |
| Degree of | 66.45 | 67.74 | −1.91 |
| coughing | | | |
| Frequency of | 64.28 | 50.38 | 27.61 |
| coughing | | | |
| Quality of sleep | 66.65 | 57.89 | 15.13 |
| Mucus discharge | 59.72 | 54.63 | 9.30 |
| in the respiratory | | | |
| tract | | | |
| Nasal congestion | 69.30 | 66.35 | 4.44 |
| Fever reduction | 89.71 | 85.38 | 5.06 |
| Composite | 69.44 | 63.83 | 8.79 |

### Example 7

### Prevention of Influenza Infection

HSRx 351, biomarker 6, and an analog of biomarker 7, 3-Hydroxyflavonone (“biomarker 7 analog”), were tested to determine the influenza viral infection prevention properties of the composition both in vitro and in vivo. It was determined that HSRx 351 prevents H1N1, H3N2, and H5N1 infection of Madin-Darby Canine Kidney Epithelial (MDCK) cells in culture and prevents viral binding to red blood cells.

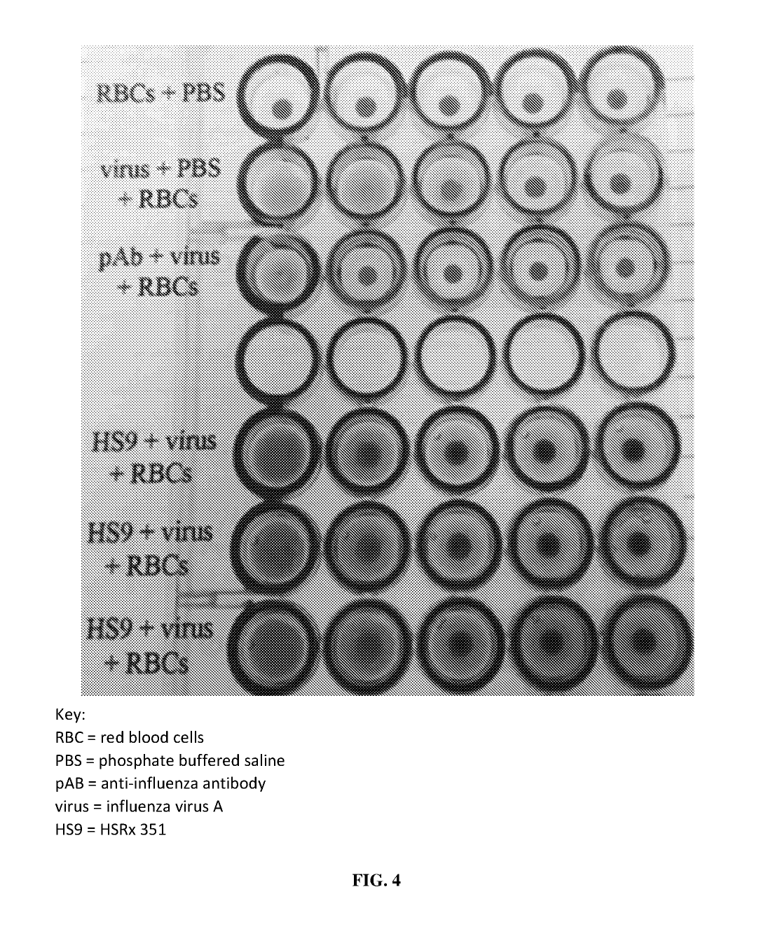

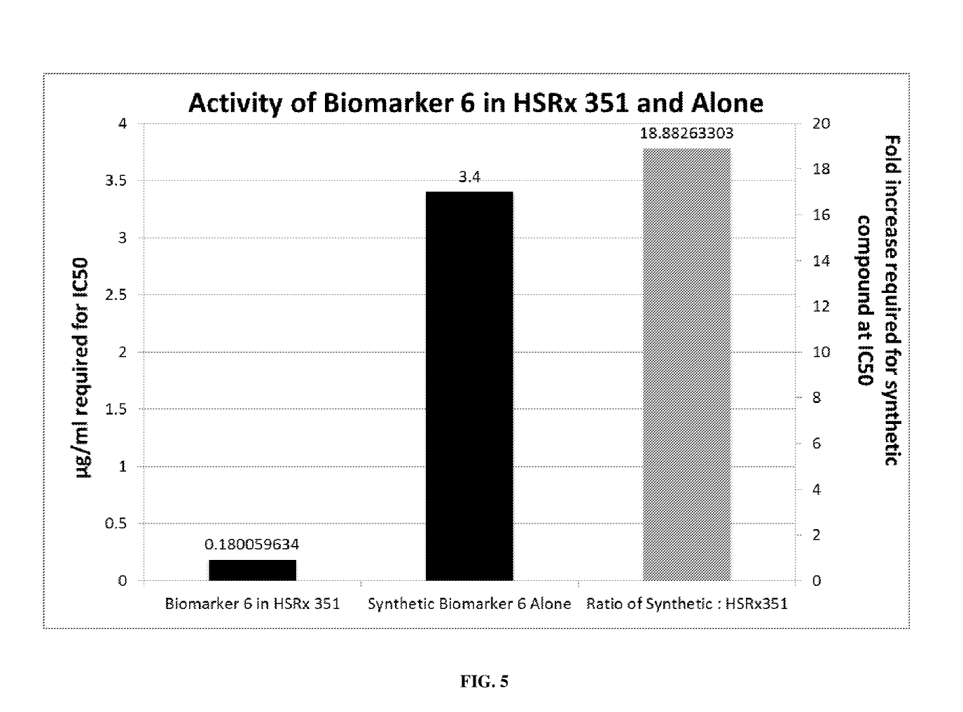

Prevention in Hemagglutination Inhibition Assay—Hemagglutination is a form of agglutination that involves the binding of red blood cells to hemagglutinin, which may be found on some viruses such as influenza virus. At high concentrations of virus, red blood cells bind the hemagglutinin protein of the virus and remain suspended in solution. At lower concentrations of virus, the red blood cells instead may settle in the bottom of the solution. Herein it has been determined that HSRx 351 prevents the binding of red blood cells to the hemagglutinin protein of influenza virus (FIG. 4).

Method: Wells were prepared with phosphate buffered saline. Serial dilutions of Influenza virus A (A/PR/8/34) (ATCC), with dilution increasing from left to right (concentration of virus is decreased from left to right), were prepared across all rows of wells except a negative control row that only contained red blood cells in phosphate buffered saline (PBS) and no virus (top row). A constant concentration of antibodies that inhibit hemagglutinin (pAB) was added to a positive control row that also contains virus (third row from top). HSRx 351 (HS9) was added at a constant concentration to three test rows (three bottom rows) to test in triplicate the ability of HSRx 351 to inhibit hemagglutination. A negative+virus control row (second row from top) was created by diluting virus in PBS at the same concentrations as the wells in the other rows, but with no addition of pAB or HSRx 351. A constant concentration of red blood cells (RBCs) was added to all wells. The level of hemagglutination was determined by visual inspection of each well. Wells wherein the red blood cells settle at the bottom to form a concentrated red dot indicate little to no hemagglutination and little to no virus binding to red blood cells. Wells wherein the red blood cells do not settle to form a concentrated red dot, but instead are dispersed in the solution indicate hemagglutination and viral binding to the red blood cells. Inhibition of hemagglutination can be determined by comparing the amount of dispersion of the red blood cells in a test well (three bottom rows) with the negative+virus control well that contains the same virus dilution (second row from the top).

Results: The negative control row (top row, RBCs+PBS) showed the inability of red blood cells to hemagglutinate in PBS alone. The negative+virus control row (second from top, virus+PBS+RBCs) showed that virus is capable of causing hemagglutination of red blood cells (see diffuse red in left two wells) but such ability is dependent on virus concentration, as decreased virus concentration decreases the amount of hemagglutination (see less diffuse red in middle well and little to no diffused red in right two wells). The positive control row (third row from top, pAb+virus+RBCs) shows that a constant concentration of pAB inhibits hemagglutination at certain concentrations of virus when compared to the negative+virus control row, see second and third well from the left. The three test rows with HSRx 351 (three bottom rows, HS9+virus+RBCs) showed that a constant concentration of HSRx 351 (HS9) inhibits hemagglutination similarly to pAB, see second and third well from the left. Thus, HSRx 351 has been shown to decrease hemagglutination and suggests that components of HSRx 351 may bind hemagglutinin of influenza virus A and prevent binding of the virus to red blood cells.

Prevention in Cell Culture Studies—A viral focus reduction infection assay was used to determine prevention of viral infection. The methods followed those described in Roschek Jr 2009. Briefly, HSRx 351, biomarker 6 analog, biomarker 7 analog, or positive controls: oseltamivir or amantadine were dissolved in EtOH and then diluted in Dulbecco's Modified Eagle Medium (DMEM). Focus forming units (FFU) of virus strains: H1N1 virus strain A/PR/8/34 (ATCC, Manassas, Va.; ATCC No. VR-1469); H3N2 (ATCC); or H5N1(ATCC); were incubated with the HSRx 351, biomarker 6 analog, biomarker 7 analog, or control dilutions for 1 hour at room temperature. The FFUs were then allowed to infect confluent MDCK cells for 1 hour at room temperature. The cells were then fixed with Formalde-fresh and permeabilized with EtOH. The FFUs were visualized using goat influenza A virus IgG polyclonal antibody (H&L) and rabbit anti-goat horseradish peroxidase conjugated affinity purified antibody (Chemicon, Temecula, Calif.) and AEC chromogen substrate (Dako, Carpinteria, Calif.).

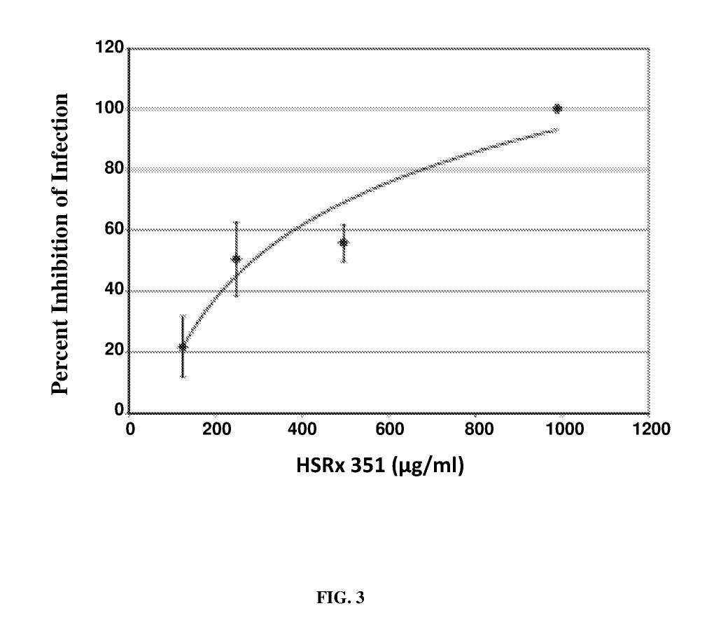

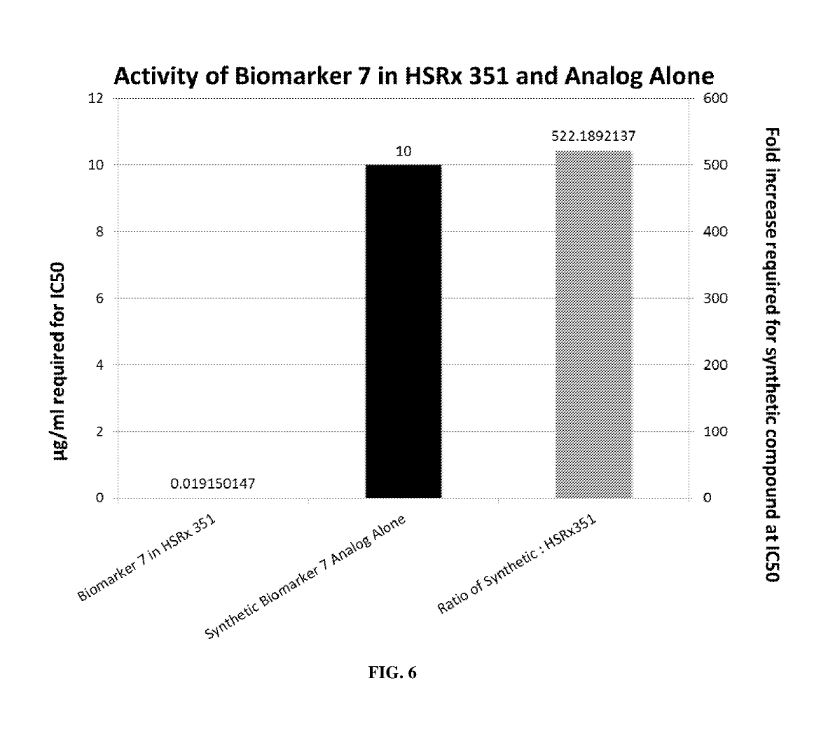

Results: Pre-incubation of virus with either HSRx 351, biomarker 6 analog, or biomarker 7 analog decreased the FFUs bound to and/or found in MDCK cells over virus not pre-exposed to the test compound/composition. In vitro HSRx 351 IC₅₀ values for the viruses were determined as show in Table 7. Further, 100% inhibition of H1N1 infection was achieved at 1,000 μg/ml (FIG. 3). Further, it was found that the activities for biomarker 6 analog and 7 analog did not account for the full activity of HSRx 351 based on the concentration of the biomarkers in HSRx 351. In fact, the HSRx 351 composition had an over 18 fold and over 500 fold higher activity than what would be expected by the concentration of Biomarker 6 or Biomarker 7 in the composition, respectively (FIG. 5 and FIG. 6). This suggests that synergy between the biomarkers of HSRx 351 may be present.

TABLE 7
Activity of HSRx 351, Biomarker 6 analog, and
Biomarker 7 analog against several influenza viruses
(adapted in part from Roschek Jr 2009).

| | | Biomarker | Biomarker | | |
|---|---|---|---|---|---|
| | | 6 analog | 7 analog | Oseltamivir | Amantadine |
| | HSRx 351 | IC₅₀ | IC₅₀ | IC₅₀ | IC₅₀ |
| | IC₅₀ | (μg/mL) | (μg/mL) | (μg/mL) | (μg/mL) |
| Virus | (μg/mL) | and (μM) | and (μM) | and (μM) | and (μM) |
| H1N1 | 252 | 0.15 and | 2.8 and | 0.1 and | 4.1 and |
| (PR) | | 0.36 | 8.7 | 0.32 | 27 |
| H3N2 | 297 | | | | |
| (Swine) | | | | | |
| H5N1 | 386 | | | | |

### Example 8

### Prevention of Influenza Infection in Combination with Tamiflu®)

Prevention in Human Patients—For the human studies, the ability of HSRx 351 in combination with Tamiflu® to prevent flu and/or flu like symptoms was evaluated. Healthy subjects were enrolled to evaluate the effect of the HSRx 351 on preventing influenza when combined with Tamiflu®. The study was a randomized, single-blind, placebo controlled, comparative therapeutic clinical study and assessment. The study showed that the combination prevented flu and/or flu like symptoms.

Methodology: Briefly, the HSRx 351 composition was formulated as a slow-dissolve lozenge containing 175 mg total of biomarkers 1 through 9 to be administered in combination with 75 mg of Tamiflu®. A randomized, single-blind, comparative therapeutic clinical study and assessment with Tamiflu® (open-label) was conducted to evaluate the efficacy of the test composition for prevention of flu and/or flu-like symptoms.

Healthy individuals who meet all of the inclusion/exclusion criteria and who twice tested negative for influenza A or B by QuickVue Influenza A+B kit (Quidell Corporation, SAN DIEGO, Calif.) at the time of the onset of the study and who did not display any other symptoms for influenza were enrolled into prevention groups C or D. Exclusion criteria included subjects less than 16 years of age and over 70 years of age, or individuals who were pregnant, breastfeeding, suffered from chronic diseases, were suspected of having a bacterial infection, participated in another clinical trial, or recently received flu medication, antiviral therapy, or influenza vaccination.

Subjects were randomized and placed on a prevention regimen of 10 days. Group C contained 30 subjects that received Tamiflu® (Oseltamivir Phosphate) alone. Group D contained 30 subjects that received Tamiflu® (Oseltamivir Phosphate) and HSRx 351. Subjects in both groups were asked to take 1 capsule containing 75 mg of Tamiflu® (Oseltamivir Phosphate) per day and subjects in group D were asked to also take two lozenges containing 175 mg each of HSRx 351 immediately following the taking of the Tamiflu® (Oseltamivir Phosphate). The treatment regimens were followed for 10 days. The first dose of medication was administered immediately after the investigator made the decision to enroll the patient into the study. After a screening visit, subjects returned for visits and evaluations on day 3, 5, and 10.

Evaluation: The efficacy of the treatment was evaluated based on the study investigator assessing symptoms and overall well-being. The symptoms assessed were: 1) aches and pains; 2) degree of coughing; 3) frequency of coughing; 4) quality of sleep; 5) mucus discharge in the respiratory tract; 6) nasal congestion; and 7) fever reduction. Symptoms were assessed at the baseline visit to determine if the two groups were clinically comparable at the start of the study. The subjects scored their symptoms from 0 to 10, 0 being no symptoms and 10 being pronounced problems, at the baseline visit and then four times a day for 10 days. Subjects also recorded any adverse events. On return visits, study personnel marked their assessment of the subject using the same scale. Additionally, a Becton Dickinson Flexible Flocked Nasal Swab was used to nasal swab the subject and to determine the presence and quantity (if any) of influenza virus infection on the first, second, third, and fourth visit (day 1, 3, 5, and 10, respectively) using a real-time polymerase chain reaction (PCR) procedure.

Statistical Analysis—Because no subjects in group D contracted an influenza virus infection, statistical comparative studies were not able to be performed.

Results: The study was conducted in what the CDC classified as a moderately severe epidemic year (2014-2015). Influenza-like illness was elevated over the national baseline of 2% for 14 consecutive weeks, peaking at 6%. QuickVue nasal swabs taken and analyzed in duplicate were all negative for patients at the beginning of the study. PCR analysis of nasal swabs taken from patients on days 1, 3, 5, and 10 confirmed that no patients in either group contracted flu over the course of the study.

Five medically related adverse events were reported in group C (Tamiflu® alone) over the course of the study, while only one medically related adverse event was reported in group D (Tamiflu® and HSRx 351).

The results show that HSRx 351 in combination with Tamiflu® can prevent flu and flu-like infection. During the study, no subject in the HSRx 351 in combination with Tamiflu® group contracted the flu. Further, HSRx 351 was shown to decrease the number of adverse events that occurred from taking Tamiflu® alone.

### Example 9

### Synergy

As previously noted, experimental results herein suggest synergism between the biomarkers disclosed herein (FIG. 5 and FIG. 6). Further, because of the predicted method of action of the biomarkers disclosed herein, it is believed that the biomarkers will act synergistically with other antiviral compounds that act through a separate mechanism. To further confirm such synergism and determine synergism with other compounds/compositions, one or more of the biomarkers disclosed herein can be tested in combination with one or more of the other biomarkers disclosed herein, and/or one or more anti-influenza drugs. Combination studies can show competitive, additive, or synergistic interactions for treatment and/or prevention of influenza viral infection, cell viability, cellular toxicity, side effects, etc. of the combination in cell culture, animal studies, human studies, etc. Non-limiting examples of studies can include those described above and herein as well as those known to one of skill in the art. As a non-limiting example, the combination of HSRx 351 and oseltamivir may be tested.

A non-limiting example of a combination assay that can be performed to determine the competitive, additive, or synergistic interactions of a combination can utilize an interaction matrix commonly used to look at drug interactions and synergy. In one instance, the interaction matrix is used in a prevention study of influenza virus infection in cell culture. Briefly, the experiment can have 25 samples: 4 with a first test compound/composition (such as HSRx 351) alone, 4 with a second test compound/composition (such as oseltamivir) alone, 1 with no chemistries, and the remaining 16 can be combinations of the first and second test compounds/compositions. 1:4 dilutions of the first test compound/composition from a starting concentration (such as 1 mg/ml for HSRx 351) and 1:4 dilutions of the second test compound/composition from a starting concentration (such as 1.0 μg/ml for oseltamivir) can be tested. The infection and culture of the influenza virus can occur in the constant presence of the inhibitory compounds. In this way, the experiment simulates a patient infected while on prophylactic treatment and tests prevention of infection by the first test compound/composition alone, the second test compound/composition alone, and the combination of the two at a range of concentrations. The data can be analyzed with the methodology of Berenbaum to determine competitive, additive, or synergistic interactions. (Berenbaum 1977).

### Example 10

### Direct Binding Assays to Influenza)

As shown above, HSRx 351 has been shown to inhibit hemagglutination using influenza virus, which is mediated solely by the viral hemagglutinin protein (FIG. 4) and suggests binding of the composition to influenza hemagglutinin. Further, direct binding of test biomarkers in HSRx 351 to influenza virus and hemagglutinin were evaluated using the methods described in Roscheck Jr 2009. It was shown that biomarker 3, 6, 7 analog, and 9 bind H1N1 virus particles and biomarkers 6 and 7 are predicted to bind hemagglutinin. The methodology used is described in Roschek Jr 2009.

Viral Particles—Briefly, H1N1 virus particles were incubated with HSRx 351 or synthetic biomarkers 6 or 7 analog, to allow binding of the compounds within HSRx 351. After incubation, unbound compounds were removed by washing the virus particles three times through a 100 kDa molecular weight cut off membrane filter (Amicon 100 kDa filter, Ultracel PL-100; Millipore Corp. Billerica, Mass.) with phosphate buffered saline (PBS). The virus particles were then collected and fixed in 100% EtOH. The fixed virus particles and the washed fractions containing the unbound chemicals were collected and analyzed directly by DART TOF-MS for comparison.

Results: Biomarkers 3, 6, 7, and 9 were identified by DART TOF-MS as significantly enriched in the fixed virus samples over the washed fractions (see Roschek Jr 2009 for biomarker 6 and biomarker 7).

Hemagglutinin—Briefly, three-dimensional free-energy minimizations using Chem 3D Ultra (Cambridgesoft, Cambridge, Mass.) molecular modeling package was employed for the free-energy minimizations of biomarkers 6 and 7 using the molecular mechanics two level of theory.

Results: Minimum free-energy modeling analysis revealed that the A and B rings of biomarkers 6 and 7 form an axis with inter-phenolic ring distances of 10.5 Å and 109 Å, respectively. This distance is well within the size constraints of the hemagglutinin (HA) binding domain pocket (14-15 Å) of influenza viruses, which is responsible for host cell receptor binding and viral entry. The phenolic regions of biomarker 6 most likely bind to the viral mannose-rich HA binding domains.

### Example 11

### Inhibition of Zika Virus

HSRx 351 was tested to determine Zika viral infection prevention properties in vitro. It was determined that HSRx 351 prevents Zika infection of African green monkey fibroblast-like kidney cells (Vero E6 cells) in culture at an IC₅₀ of less than 100 μg/ml and approximately 80 μg/ml.

Zika Prevention in Cell Culture Studies—A viral plaque reduction neutralization test (PRNT) was used to determine prevention of viral infection. Briefly, HSRx 351 was dissolved in 200 μl DMSO and then diluted in Dulbecco's Modified Eagle Medium (DMEM), pH 7.2. Plaque forming units (PFU) of Zika virus strain were incubated with the HSRx 351 or control dilutions for 1 hour at room temperature. The PFUs were then allowed to infect confluent Vero cells for 1 hour at room temperature. The plaques were visualized by staining with neutral red.

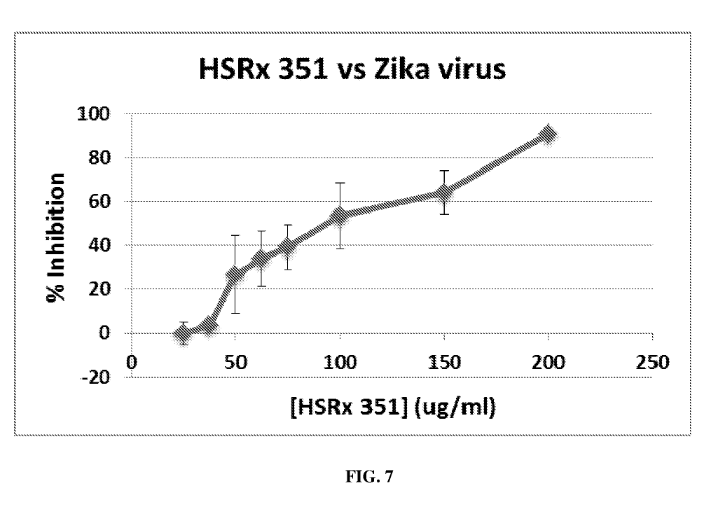

Results: Pre-incubation of virus with HSRx 351 decreased the plaque forming units (PFUs) bound to and/or found in Vero E6 cells over virus not pre-exposed to the test composition. See FIG. 7. In vitro HSRx 351 IC₅₀ values for Zika virus was determined to be approximately 80 μg/ml. Further, 100% inhibition of Zika infection was achieved at 250 μg/ml.

### Example 12

### Inhibition of other Enveloped Viruses

HSRx 351 was tested to determine viral infection prevention properties in vitro for the enveloped viruses HIV (multiple subtypes), herpes simplex 1 (HSV-1), and dengue (DEN-2). It was determined that HSRx 351 prevents infection of cells in culture at an IC₅₀ for each of the viruses as shown in Table 8. These results, combined with the additional binding, treatment, prevention, and inhibition results of other envelope viruses, surprisingly show that the compositions disclosed herein are broad spectrum anti-viral compositions against enveloped viruses.

Infection Prevention in Cell Culture Studies—A viral focus reduction infection assay was used to determine prevention of viral infection. Briefly, HSRx 351 was dissolved in 100% DMSO and then diluted in Dulbecco's Modified Eagle Medium (DMEM). Plaque forming units (PFU) or focus forming units (FFU) of the test virus strains were incubated with the HSRx 351 or control dilutions for 1 hour at room temperature. The PFUs/FFUs were then allowed to infect for 1 hour at room temperature confluent GHOST cells for HIV (see Fink et al., 2009 for experimental conditions), Vero cells for HSV-1, or LLCMK2 cells for dengue virus. The cells were then fixed with Formalde-fresh and permeabilized with EtOH. The FFUs in dengue infected cells were visualized using goat IgG polyclonal antibodies against the dengue virus (H&L) and rabbit anti-goat horseradish peroxidase conjugated affinity purified antibody (Chemicon, Temecula, Calif.) and AEC chromogen substrate (Dako, Carpinteria, Calif.). Light microscopy was used to determine infection rates for HSV-1. Fluorescent microscopy was used to determine infection rates for HIV.

Results: Pre-incubation of virus with HSRx 351 decreased the FFUs and PFUs bound to and/or found in cells over virus not pre-exposed to the test composition. In vitro HSRx 351 IC₅₀ values for HIV-1 subtypes B1, B2, C1, and C2 were determined to range from 201 μg/m1 to 36 μg/ml. In vitro HSRx 351 IC₅₀ values for HSV-1 was 40 μg/m1 and for DEN-2 it was 63 μg/ml.

TABLE 8
Activity of HSRX 351 against several envelope viruses.

| | HSRx 351 |
|---|---|
| | IC₅₀ |
| Virus | (μg/mL) |
| HIV-1 sub B1 | 70 |
| HIV-1 sub B2 | 201 |
| HIV-1 sub C1 | 36 |
| HIV-1 sub C2 | 169 |
| HSV-1 | 40 |
| DEN-2 | 63 |

### Example 13

### Direct Binding Assays to Dengue Virus

As shown above, HSRx 351 has been shown to inhibit dengue virus and suggests binding of the composition to dengue. Further, direct binding of HSRx 351 to dengue virus was evaluated. It was shown that several compounds in HSRx 351 bind dengue virus in vitro.

Viral Particles—Briefly, dengue virus particles (DEN-2) were incubated with HSRx 351, to allow binding of the compounds within HSRx 351. After incubation, unbound compounds were removed by washing the virus particles three times through a 100 kDa molecular weight cut off membrane filter (Amicon 100 kDa filter, Ultracel PL-100; Millipore Corp. Billerica, Mass.) with phosphate buffered saline (PBS). The virus particles were then collected and fixed in 100% EtOH. The fixed virus particles and the washed fractions containing the unbound chemicals were collected and analyzed directly by DART TOF-MS for comparison.

Results: Biomarkers 3, 6, 7, and 9 were identified by DART TOF-MS as significantly enriched in the fixed virus samples over the washed fractions.

### Example 14

### Inhibition of Human Rhinovirus

HSRx 351 was tested to determine viral infection prevention properties in vitro for human rhinovirus (HRV) a non-enveloped virus that commonly infects humans and is associated with the common cold. It was determined that HSRx 351 prevents HRV-16 infection HeLa cells in culture at an IC₅₀ of 90 μg/ml. These results, along with the influenza and influenza-like illness treatment and prevention, inhibition and binding studies for influenza viruses surprisingly show that compositions disclosed herein can be used to treat and prevent influenza and influenza-like illnesses.

Human Rhinovirus Prevention in Cell Culture Studies—A viral focus reduction infection assay was used to determine prevention of viral infection. Briefly, HSRx 351 was dissolved in 100% DMSO and then diluted in Dulbecco's Modified Eagle Medium/F12 (DMEM/F12), pH 7.2. Plaque forming units (PFU) of HRV-16 virus strain were incubated with the HSRx 351 or control dilutions for 1 hour at room temperature. The PFUs were then allowed to infect 80% confluent HeLa cells for 1 hour at room temperature. The cells were then fixed with Formalde-fresh and permeabilized with EtOH. The PFUs were visualized using goat HRV virus IgG polyclonal antibody (H&L) and rabbit anti-goat horseradish peroxidase conjugated affinity purified antibody (Chemicon, Temecula, Calif.) and AEC chromogen substrate (Dako, Carpinteria, Calif.).

Results: Pre-incubation of virus with HSRx 351 decreased the PFUs bound to and/or found in HeLa cells over virus not pre-exposed to the test compound/composition. In vitro HSRx 351 IC₅₀ values for Human Rhinovirus was determined to be 90 μg/ml.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Cumulative Number of Confirmed Human Cases of Avian Influenza A/(H5N1) Reported to WHO. May 6, 2009. [cited 2009 June 17]. http://www.who.int/csr/disease/avian_influenza/country/cases_table_2009_05_06/en/inde x.html
Dengue: Center for Disease Control and Prevention. Jan. 19, 2016 [cited Aug. 18, 2016]. https://www.cdc.gov/idengue/
Epidemic and Pandemic Alert and Response (EPR): World Health Organization. Influenza A(H1N1). Update 58. Jul. 6, 2009. [cited 2009 Jul. 23].

http://www.who.int/csr/don/2009_07_06/en/index.html
FDA Approves Rapivab to Treat Flu Infection: U.S. Food and Drug Administration. Dec.22, 2014.[cited 2015 June 8]. http://www.fda.gov/NewsEvents/Newsroom/PressAnnouncements/ucm427755.htm
Influenza: Center for Disease Control and Prevention. Apr. 29, 2015[cited 2015 May 22]. http://www.cdc.gov/nchs/fastats/flu.htm
Influenza (Flu): Center for Disease Control and Prevention. Key facts about seasonal flu vaccine. Oct. 22, 2014[cited 2015 May 22]. http://www.cdc.gov/flu/protect/keyfacts.htm
Global Alert and Response (GAR): World Health Organization. Influenza A(H1N1). Pandemic (H1N1) 2009 —update 101. May 21, 2010. [cited 2015 May 22]. http://www.who.int/csr/don/2010_05_21/en/
Relenza: Consumer Questions and Answers: U.S. Food and Drug Administration. Jan. 25, 2013. [cited 2015 May 22]. http://www.fda.gov/Drugs/DrugSafety/PostmarketDrugSafetyInformationforPatientsandP roviders/ucm188870.htm, accessed May 22, 2015
Zika Virus: Center for Disease Control and Prevention. Aug. 16, 2016[cited 2016 Aug. 17]. http://www.cdc.gov/zika/index.html
Beigel J H. (2005) Avian Influenza A (H5N1) Infection in Humans. New England Journal of Medicine 353:1374-1386.
Belshe R B, Burk B, Newman F, Cerruti R L and Sim I S. (1989) Resistance of influenza A virus to amantadine and rimantadine: results of one decade of surveillance. Journal of Infectious Diseases 159:430-435.
Berenbaum M C. (1977) Synergy, additivism and antagonism in immunosuppression. A critical review. Clin Exp. Immunol. 28(1): 1-18.
Cody R B, Laramee J A and Durst H D. (2005) Versatile new ion source for the analysis of materials in open air under ambient conditions. Analytical Chemistry 77:2297-2302.
Dawood F S, Iuliano A D, Reed C, Meltzer M I, Shay D K, Cheng P Y, Bandaranayake D, Breiman R F, Brooks W A, Buchy P, Feikin D R, Fowler K B, Gordon A, Hien N T, Horby P, Huang Q S, Katz M A, Krishnan A, Lal R, Montgomery J M, Mølbak K, Pebody R, Presanis A M, Razuri H, Steens A, Tinoco Y O, Wallinga J, Yu H, Vong S, Bresee J, Widdowson M A. (2012) Estimated global mortality associated with the first 12 months of 2009 pandemic influenza A H1N1 virus circulation: a modelling study. Lancet Infectious Diseases 12(9): 687-695.

Fields B N, Knipe D M and Howley P M (2001) Fields Virology: in Orthomyxoviruses, 1533-1580. Lippincott, Williams & Wilkins, Philadelphia.
Fink R, Roschek Jr B and Alberte R S. (2009) HIV type-1 entry inhibitors with a new mode-of-action. Antiviral Chemistry and Chemotherapy 19(6):243-255.
Hayden F G. (1994) Amantadine and rimantadine resistance in influenza A viruses. Current Opinion in Infectious Diseases 7:674-677.
Jefferson T, Demicheli V, Rivetti D, Jones M, Di Pietrantonj C and Rivetti A. (2006) Antivirals for influenza in healthy adults: systematic review. Lancet 367:303-313.
Kong F. (2009) Pilot clinical study on a proprietary Elderberry extract: efficacy in addressing influenza symptoms. Online Journal of Pharmacology and PharmacoKinetics 5:32-43.
Le Q M, Kiso M, Someya K, Sakai Y T, Nguyen T H, Nguyen K H, Pham N D, Ngyen H H Yamada S, Muramoto Y, Horimoto T, Takada A, Goto H, Suzuki T, Suzuki Y and Kawaoka Y. (2005) Avian flu: isolation of drug-resistant H5N1 virus. Nature 437:1108.
Makela M J, Pauksens K, Rostila T, Fleming D M, Man C Y, Keene O N and Webster A. (2000) Clinical efficacy and safety of the orally inhaled neuraminidase inhibitor zanamivir in the treatment of influenza: a randomized, double-blind, placebo-controlled European study. Journal of Infection 40:42-48.
Monto A S, Fleming D M, Henry D, de Groot R, Makela M, Klein T, Elliott M, Keene O N and Man C Y. (1999) Efficacy and safety of the neuraminidase inhibitor zanamivir in the treatment of influenza A and B virus infections. Journal of Infectious Diseases 180:254-261.
Moscona A. (2005) Oseltamivir resistance—disabling our influenza defenses. New England Journal of Medicine 353:2633-2636.
Nagai T, Miyaichi Y, Tomimori T, Suzuki Y and Yamada H. (1990) Inhibition of influenza virus sialidase and anti-influenza virus activity by plant flavonoids. Chemical & Pharmaceutical Bulletin 38:1329-1332.
Nicholson K G, Webster R G, Hay A J, eds. (1998) Textbook of influenza. Oxford: Blackwell Science.
Nicholson K G, Aoki F Y, Osterhaus A D, Trottier S, Carewicz O, Mercier C H, Rode A, Kinnersley N and Ward P. (2000) Efficacy and safety of oseltamivir in treatment of acute influenza: a randomised controlled trial. Neuraminidase Inhibitor Flu Treatment Investigator Group. Lancet 355:1845-1850.
Roschek Jr. B, and Alberte R S. (2009) Pharmacokinetics of cyaniding and anti-influenza phytonutrients in an elder berry extract determined by LC-MS and DART TOF-MS. Online Journal of Pharmacology and Pharmacokinetics 4:1-17.
Roschek Jr. B, Fink R C, McMichael M D, Li D and Alberte R S. (2009) Elderberry flavonoids bind to and prevent H1N1 Infection in vitro. Phytochemistry 70(10):1255-1261.
Roxas M and Jurenka J. (2007) Colds and influenza: a review of diagnosis and conventional, botanical, and nutritional considerations. Alternative Medicine Review 12:25-48.
Subbarao K, Murphy B R and Fauci A S. (2006) Development of effective vaccines against pandemic influenza. Immunity 24:5-9.
Thompson W W, Shay D K, Weintraub E, Brammer L, Cox N, Anderson L J and Fukuda K. (2003) Mortality associated with influenza and respiratory syncytial virus in the United States. Journal of the American Medical Association 289:179-186.
Trifonov, V, Khiabanian, H, and Rabadan, R.(2009) Geographic dependence, surveillance, and origins of the 2009 influenza A (H1N1) virus. New England Journal of Medicine. DOT: 10.1056/nejmp0904572.
Vivek S, et al. (2009) Triple-Reassortant Swine Influenza A (H1) in Humans in the United States, 2005-2009. New England Journal of Medicine 360. DOI: 10.1056/NEJMoa0903812.
von Itzstein M, Wu W-Y, Kok G B, Pegg M S, Dyason J C, Jin B, Phan T V, Smythe M L, White H F, Oliver S W, Colman P M, Varghese J N, Ryan D M, Woods J M, Bethell R C,
Hotham V J, Cameron J M and Penn C R. (1993) Rational design of potent sialidase-based inhibitors of influenza virus replication. Nature 363:418-423.
Wang C, Takeuchi K, Pinto L H and Lamb R A. (1993) Ion channel activity of influenza A virus M2 protein: characterization of the amantadine block. Journal of Virology 67:5585-5594.
Webster R G, Bean W J, Gorman O T, Chambers T M and Kawaoka Y. (1992) Evolution and ecology of influenza A viruses. Microbiological Reviews 56:152-179.
Zakay-Rones Z, Varsano N, Zlotnik M, Manor O, Regev L, Schlesinger M and Mumcuoglu M. (1995) Inhibition of several strains of influenza virus in vitro and reduction of symptoms by an elderberry extract (Sambucus nigra L.) during an outbreak of influenza B Panama. Journal of Alternative and Complementary Medicine 1:361-369.
Zakay-Rones Z, Thom E, Wollan T and Wadstein J. (2004) Randomized study of the efficacy and safety of oral elderberry extract in the treatment of influenza A and B virus infections. Journal of International Medical Research 32:132-140. US 2009/0149530

1. A composition comprising any one of, or any combination of, the following biomarkers:
(a) biomarker 1 having an accurate mass of 112.027 amu and having a relative abundance of at least 2.36%;
(b) biomarker 2 having an accurate mass of 126.032 amu and having a relative abundance of at least 33.26%;
(c) biomarker 3 having an accurate mass of 155.095 amu and having a relative abundance of at least 1.86%;
(d) biomarker 4 having an accurate mass of 160.087 amu and having a relative abundance of at least 5.03%;
(e) biomarker 5 having an accurate mass of 166.099 amu and having a relative abundance of at least 9.26%; or
(f) biomarker 8 having an accurate mass of 507.342 amu and having a relative abundance of at least 0.60%,
wherein each biomarker is found in Sambucus nigra, and
wherein the relative abundance is relative abundance as compared to 0.01 mg/ml curcumin spiked in 1 mg/ml of the composition.

2. The composition of claim 1, having at least 2, 3, 4, 5, or all of biomarkers 1 to 5 and 8.

3. The composition of any one of claims 1 to 2, wherein the composition further comprises any one of, or any combination of, or all of the following additional biomarkers:
(g) biomarker 6 having an accurate mass of 358.146 amu;
(h) biomarker 7 having an accurate mass of 478.295 amu;
(i) biomarker 9 having an accurate mass of 606.436 amu,
wherein each biomarker is found in Sambucus nigra.

4. The composition of claim 3, further comprising:
(j) biomarker 6 having a relative abundance of at least 11.37%;
(k) biomarker 7 having a relative abundance of at least 1.20%; and
(l) biomarker 9 having a relative abundance of at least 0.07%,
wherein the relative abundance is relative abundance as compared to 0.01 mg/ml curcumin spiked in 1 mg/ml of the composition.

5. The composition of claim 1, further comprising:
(a) biomarker 1 having a relative abundance of between 2.36% and 6.94%;
(b) biomarker 2 having a relative abundance of between 33.26% and 85.75%;
(c) biomarker 3 having a relative abundance of between 1.86% and 4.69%;
(d) biomarker 4 having a relative abundance of between 5.03% and 12.89%;
(e) biomarker 5 having a relative abundance of between 9.26% and 24.11%;
(f) biomarker 8 having a relative abundance of between 0.60% and 1.75%,
wherein the relative abundance is relative abundance as compared to 0.01 mg/ml curcumin spiked in 1 mg/ml of the composition.

6. The composition of claim 4, further comprising:
(j) biomarker 6 having a relative abundance of between 11.37% and 31.81%;
(k) biomarker 7 having a relative abundance of between 1.20% and 3.40%; and
(l) biomarker 9 having a relative abundance of between 0.07% and 1.38%,
wherein the relative abundance is relative abundance as compared to 0.01 mg/ml curcumin spiked in 1 mg/ml of the composition.

7. The composition of any of claims 1 to 6 wherein the mass of each biomarker is the mass as determined by a Direct Analysis in Real Time-TOF (DART-TOF) mass spectrometer.

8. The composition of any one of claims 1 to 7, wherein at least one of biomarkers 1 through 9 are synthetically obtained.

9. The composition of any one of claims 1 to 7, wherein at least one of biomarkers 1 through 9 are obtained from an organism.

10. The composition of claim 9, wherein at least one of biomarkers 1 through 9 are obtained from Sambucus nigra fruit.

11. The composition of any one of claims 1 to 10, wherein the composition has an at least 90%, preferably at least 95%, or at least 98% batch-to-batch chemical consistency of relative abundance for the biomarkers.

12. The composition of any one of claims 1 to 11, wherein the composition further comprises an anti-viral drug.

13. The composition of claim 12, wherein the composition further comprises an anti-influenza drug.

14. The composition of claim 13, wherein the anti-influenza drug is oseltamivir, zanamivir, rimantadine, amantadine, peramivir, or salts thereof, or any combination thereof.

15. The composition of claim 14, wherein the anti-influenza drug is oseltamivir, a salt thereof, or any combination thereof.

16. The composition of any one of claims 1 to 15, wherein the composition is formulated for oral administration.

17. The composition of claim 16, wherein the composition is one or more of a lozenge, a powder, a tablet, a gel-cap, a delayed release capsule, a quick release capsule, a gelatin, a liquid solution, and/or a dissolvable film.

18. The composition of any one of claims 1 to 15, wherein the composition is formulated for topical application, intravenous administration, and/or intranasal delivery.

19. The composition of any one of claims 1 to 18, wherein the composition has an IC₅₀ lower than 500 μg/m1 against influenza virus.

20. The composition of any of claims 1 to 19, wherein at least one of biomarkers 1 to 5 and 8 is capable of binding to an influenza virus and blocking influenza viral entry into a cell.

21. The composition of claim 20, wherein the at least one of biomarkers 1 to 5 and 8 is capable of binding hemagglutinin of the influenza virus.

22. A method of treating a subject having influenza and/or an influenza-like illness, the method comprising administering any one of the compositions of claims 1 to 21 to the subject, wherein the subject is treated.

23. The method of claim 22, wherein the subject has a fever, a headache, muscle aches, coughing, mucus discharge, or nasal congestion, or any combination thereof.

24. The method of any one of claims 22 to 23, wherein the subject has influenza and is infected with an Influenzavirus A and/or an Influenzavirus B virus.

25. The method of claim 24, wherein the influenza virus is H1N1, H3N2, H3N5, H5N1, and/or Influenza B virus.

26. The method of any one of claims 22 to 23, wherein the subject has an influenza-like illness and is infected with rhinovirus.

27. The method of any one of claims 22 to 26, wherein the subject is administered a total sum of between 1 and 5,000 mg, preferably between 10 and 1,500 mg, between 50 and 1,000 mg, or between 100 and 500 mg of the biomarker(s) during a 24 hour period.

28. The method of any one of claims 22 to 27, wherein the composition is administered at least once a day for at least three days.

29. A method of treating a subject infected with an envelope virus, the method comprising administering any one of the compositions of claims 1 to 21 to the subject, wherein the subject is treated.

30. The method of claim 29, wherein the subject is infected with a HIV, herpes complex virus, flavivirus virus, Influenzavirus A virus, and/or Influenzavirus B virus.

31. The method of any one of claims 29 to 30, wherein the subject is infected with a flavivirus virus and the flavivirus virus is Zika virus and/or dengue virus.

32. The method of claim 31, wherein the subject is infected with Zika virus.

33. The method of any one of claims 29 to 32, wherein the subject is administered a total sum of between 1 and 5,000 mg, preferably between 10 and 1,500 mg, between 50 and 1,000 mg, or between 100 and 500 mg of the biomarker(s) during a 24 hour period.

34. The method of any one of claims 29 to 33, wherein the composition is administered at least once a day for at least three days.

35. A method of preventing influenza and/or an influenza-like illness in a subject, the method comprising administering any one of the compositions of claims 1 to 21 to the subject, wherein influenza and/or an influenza-like illness is prevented.

36. The method of claim 35, wherein influenza caused by an Influenzavirus A and/or an Influenzavirus B virus is prevented.

37. The method of claim 36, wherein the influenza virus is H1N1, H3N2, H3N5, H5N1, and/or Influenza B virus.

38. The method of claim 35, wherein influenza-like illness caused by a Rhinovirus is prevented.

39. The method of any one of claims 35 to 38, wherein the subject is administered a total sum of between 1 and 5,000 mg, preferably between 10 and 1,500 mg, between 50 and 1,000 mg, or between 100 and 500 mg of the biomarker(s) during a 24 hour period.

40. The method of any one of claims 35 to 39, wherein the composition is administered at least once a day for at least three days.

41. A method of preventing an envelope virus infection in a subject, the method comprising administering any one of the compositions of claims 1 to 21 to the subject, wherein an envelope virus infection is prevented.

42. The method of claim 41, wherein the envelope virus infection that is prevented is an infection by a HIV, herpes complex virus, flavivirus virus, influenzavirus A virus, and/or influenzavirus B virus.

43. The method of any one of claims 41 to 42, wherein a flavivirus virus infection is prevented and the flavivirus virus is Zika virus and/or dengue virus.

44. The method of claim 43, wherein a Zika virus infection is prevented.

45. The method of any one of claims 41 to 44, wherein the subject is administered a total sum of between 1 and 5,000 mg, preferably between 10 and 1,500 mg, between 50 and 1,000 mg, or between 100 and 500 mg of the biomarker(s) during a 24 hour period.

46. The method of any one of claims 41 to 45, wherein the composition is administered at least once a day for at least three days.

47. A method of producing a composition of any of claims 1 through 21, wherein the method of producing produces a composition having an at least 90%, preferably at least 95% or at least 98% batch-to-batch chemical consistency of relative abundance for the biomarkers.
